(19) United States
(12) Patent Application Publication
(10) Pub. No.: US 2008/0096838 A1
(43) Pub. Date: Apr. 24, 2008

(54) Guanosine rich oligonucleotides and methods of inducing apoptosis in tumor cells

(76) Inventors: Eric B. Kmiec, Landenberg, PA (US); Hetal Parekh-Olmedo, Mickleton, NJ (US); Timothy Schwartz, Newark, DE (US)

Correspondence Address: MCCARTER & ENGLISH, LLP;BASIL S. KRIKELIS, Renaissance Centre, 405 N. King Street, 8th Floor, WILMINGTON, DE, 19801, US

(21) Appl. No.: 11/890,595

(22) Filed: Aug. 7, 2007

Related U.S. Application Data
(60) Provisional application No. 60/836,323, filed on Aug. 8, 2006.
(60) Provisional application No. 60/920,353, filed on Mar. 27, 2007.

(51) Int. Cl. A61K 31/70; A61P 43/00; C07H 21/04; C12Q 1/68

(52) U.S. Cl. 514/44; 435/6; 536/22.1

(57) ABSTRACT

Presently described is a guanosine-rich polynucleotide molecule with therapeutic utility for treating or preventing the growth of cancerous cells. In addition, a method of retarding cell cycle progression and inducing apoptosis in tumor cells using synthetic oligonucleotides is also described.

### CROSS-REFERENCE TO RELATED APPLICATIONS

Under 35 U.S.C. § 119(e) this application claims the benefit of U.S. Provisional Applications No. 60/836,323 filed Aug. 8, 2006, and 60/920,353 filed Mar. 27, 2007, which are hereby incorporated by reference in their entirety.

### INCORPORATION BY REFERENCE

In compliance with 37 C.F.R. § 1.52(e)(5), the sequence information contained on compact disc, file name: 99689-00047.txt; size 4 KB; created on: 7 Aug. 2007; using PatentIn-3.3, is hereby incorporated by reference in its entirety. The Sequence Listing information recorded in computer readable form (CRF) is identical to the written Sequence Listing provided herewith. The data in the paper copy of the Sequence Listing, and Computer Readable Form of the Sequence Listing submitted herewith contain no new matter, and are fully supported by the priority applications.

### FIELD OF THE INVENTION

The invention relates to the field of synthetic oligonucleotides and methods for their use in the treatment of diseases, for example, cancer.

### BACKGROUND

Guanosine (G)-rich DNA and RNA form inter- and intramolecular four-stranded structures known as G-quartets. G-quartets are formed when four G-bases are associated into a cyclic Hoogsten H-bonding arrangement wherein each G-base makes two H-bonds with its neighboring G-base. Ultimately, G-quartets stack on top of each other, giving rise to tetrad-helical structures. The stability of these G-quartets is related to several factors, including the presence of monovalent cations such as K⁺ and Na⁺, the concentration of G-rich oligonucleotides present, and the sequence of the G-rich oligonucleotides being used.

G-rich oligonucleotides (GROs) display effective antiproliferative activity when added to cancer cell lines. It has been reported that treatment of tumor cells with GROs inhibits cell cycle progression by interfering directly with DNA replication. Researchers in the art have studied GRO quadruplexes to determine whether there are any features associated with the GRO that are behind the antiproliferative activity in the hopes of facilitating design of effective, cancer-treating GROs. The results to date, however, suggest that there is no simple relationship between the structure of GRO quadruplexes, their biophysical properties and their antiproliferative activity. If fact, structure/function data indicate that the GRO-protein interaction is highly selective and sensitive to perturbations in the structure of the GRO backbone (i.e., the precise chemical composition of the GRO). (See Bates et al., Antiproliferative activity of G-rich oligonucleotides correlates with protein binding, J. Biol. Chem., 274, 26369-26377 (1999); Xu et al., Inhibition of DNA replication and induction of S phase cell cycle arrest by G-rich oligonucleotides, J. Biol. Chem., 276, 43221-43230 (2001); Dapic et al., Antiproliferative activity of G-quartet-forming oligonucleotides with backbone and sugar modifications, Biochemistry, 41, 3676-3685 (2002); and Dapic et al., Biophysical and biological properties of quadruplex oligodeoxyribonucleotides, Nucl. Acids Rsch, 31, 8: 2097-2107 (2003), the teachings of which are incorporated by reference in their entirety.).

Current cancer chemotherapies promote cancer cell death and inhibit cancer cell growth, however, these chemotherapies are highly toxic to cancer patients and their administration results in a multitude of unpleasant and unbearable side effects While some GROs have been demonstrated to be capable of inhibiting cancer cell proliferation, to date, none have be shown to induce cancer cell apoptosis as well. Obviously, a cancer treatment that promotes cancer cell death, inhibits cancer cell growth, and is largely non-toxic to cancer patients is highly desirable.

### SUMMARY

The present invention relates to the discovery that guanosine-rich oligonucleotides (GROs) are capable of inducing profound effects on cancer cell cycle progression and viability. In particular, the invention relates to certain GROs that demonstrate the ability to selectively interrupt the cell cycle and induce apoptosis in cancer cells, for example, esophageal cancer cells. While not being limited to any particular theory it is believed that the activity of the GROs of the invention are mediated through interactions with inter- or intracellular protein or nucleic acid targets.

Thus in one aspect, the invention is directed to a composition comprising a polynucleotide or nucleic acid comprising approximately 19-21 guanosine residues in length. In an embodiment of this aspect, the invention relates to a nucleic acid molecule comprising the polynucleotide of SEQ ID NO. 1. The GRO of the invention can be incorporated into a pharmaceutically acceptable form, and can also be combined with one or more pharmaceutically acceptable carriers, excipients or adjuvants.

In another aspect, the invention relates to a methods of treating cancer comprising administering a composition comprising a DNA molecule comprising the sequence set forth in SEQ ID NO. 1.

Additional aspects and advantages of the invention will be evident to those of skill in the art in view of the instant drawings, detailed description, examples, and appended claims. These additional aspects and advantages are expressly included within the scope of the present invention.

### BRIEF DESCRIPTION OF THE DRAWINGS

FIG. 1. Is a comparative bar graph demonstrating the relative cell population of three cell cycle phases for esophageal carcinoma cells. When no oligonucleotide was added to the cells,

FIG. 2. Is a flow cytometry profile depicting a cell cycle which shows two distinct peaks of cell populations, the first peak being the G1 phase of the cell cycle and the second peak being the G2 phase.

FIG. 3. A. Secondary structure of C20 (SEQ ID NO. 6), A20 (SEQ ID NO. 7), G20 (SEQ ID NO. 1), and T20 (SEQ ID NO. 8) ODNs. CD spectra (performed on AVIV Circular Dichroism Spectrometer Model 215 at 24° C. in KCl; ODNs at 15 μM) is shown for the ODN nucleotide sequences C20 (SEQ ID NO. 6) in blue, A20 (SEQ ID NO. 7) in red, G20 (SEQ ID NO. 1) in green, and T20 (SEQ ID NO. 8) in violet. B. Transfection of synthetic ODNs in OE19 cells. OE19 cells were electroporated with 10 μM of FAM-labelled non-specific nucleotide and analyzed by FACS 24 hrs later for uptake efficiency, using propidium iodide to distinguish dead cells. Confocal microscopy was used to observe the cellular compartmentalization of TAMRA-labelled G20 nucleotide (red) 24 hrs post-electroporation; DAPI nuclear stain is shown in blue.

FIG. 4. A. Cell cycle profiles of untreated OE19 cells. Cell cycle for OE19 esophageal cancer cells was analyzed by FACS at 24, 48, 72, and 96 hrs; DNA content is quantified by propidium iodide staining. B. Cell cycle profiles of OE19 cells treated with G20 ODN (10 μM). Cell cycle for OE19 esophageal cancer cells was analyzed by FACS at 24, 48, 72, and 96 hrs, with DNA content quantitated by propidium iodide staining. C. Cell cycle profiles of OE19 cells treated with monomeric ODNs at 96 hrs. Cell cycle for OE19 esophageal cancer cells was analyzed by FACS at 96 hrs after treatment with G20, A20, T20 (10 μM). DNA content was quantitated by propidium iodide staining.

FIG. 5. G20 ODN demonstrates dose-dependent response in OE19 cells. Cell cycle for OE19 esophageal cancer cells treated with increasing levels of G20 was analyzed by FACS at 96 hrs.

FIG. 6. A. Evidence for apoptosis in treated OE19 cells. Caspase-3,7 activity was assessed via luminescent assay in OE19 cells 96 hrs after treatment. Cells were treated with either non-specific ODN (10 μM), no ODN, or untreated cells and levels of caspase activity are determined per 5×10⁴ cells. B. Assessment of nuclear fragmentation in OE19 cells. Chromatin condensation and nuclear fragmentation was visualized by Hoechst staining in OE19 cells 96 hrs after treatment. Cells were treated with either non-specific ODN(10 μM), no ODN, or untreated cells and imaged at 40× magnification.

FIG. 7. A. Cell cycle profiles of treated Het-1A cells. Cell cycle for Het-1A non-cancerous esophageal cells was analyzed by FACS at 96 hrs. Cells were untreated or treated with or without 10 μM G20 and visualized. B. Cell cycle profiles of treated MRC5 cells. Cell cycle for MRC5 non-cancerous lung cells that were untreated or treated with or without 10 μM G20 (SEQ ID NO. 1) was analyzed by FACS at 96 hrs.

FIG. 8. A. Cell cycle profiles for OE19 cells treated with alternate G-quadruplex-forming ODNs. Cell cycle for OE19 esophageal cancer cells was analyzed by FACS at 96 hrs, treated with the quadruplex-forming G-rich ODNs T30923 (SEQ ID NO. 4) and T40216 (SEQ ID NO. 5), at concentrations of 10 μM or 20 μM, respectively. B. Secondary structure of T30923, T40216, and G20 (SEQ ID NO. 1) ODNs. CD spectra (24° C. in KCl) is shown for the ODN nucleotide sequences T30923 in red, T40216 in blue, and the G20 in green. Samples were evaluated by AVIV Circular Dichroism Spectrometer Model 215 at 24° C. in KCl.

FIG. 9. G20-Biotin pull-down immunoblots in OE19 (malignant esophageal adenocarcinoma cell line).

FIG. 10. G20-Biotin pull-down immunoblots in Het-1A (non-malignant human esophageal cell line).

FIG. 11. Mean tumor volume by treatment group. Average tumor volume (mm³) vs. time (days) for Vehicle Control, Nonsense, and Sense subjects. Tumor volume was measured at intervals of approximately 48 hrs.

FIG. 12. Change in tumor volume by group. Average change in tumor volume (%) vs. time (days) for Vehicle Control, Nonsense, and Sense subjects, (error bars=SEM). Percent change in tumor volume is displayed at intervals of approximately 48 hrs.

FIG. 13. Survival proportions per treatment group. Percent survival (%) vs. time (days) for Vehicle Control, Nonsense, and Sense treatment groups. Survival represents the period of time before tumors reached 10% volume to body weight or 1.5 cm for each individual subject, after which subjects were sacrificed.

FIG. 14. Exponential growth curves for mean tumor volume by treatment group. Average tumor volume (mm³) vs. time (days) for Vehicle Control, Nonsense, and Sense subjects. Growth curves represent rate of tumor volume increase for the first 12 days of treatment. The sense oligo (G20) has resulted in a decrease in tumor growth rate up to day 15 post treatment at which point the sacrifice of mice that have reached maximum tumor burden in the control and anti-sense groups skews the data. The analysis of ‘time to maximum tumor burden’ shows this same effect.

### DETAILED DESCRIPTION

While the specification concludes with claims particularly pointing out and distinctly claiming the subject matter regarded as forming the present invention, it is believed that the invention will be better understood from the following preferred embodiments of the invention taken in conjunction with the accompanying drawings.

Short synthetic oligonucleotides (ODN) can be used to block cellular processes involved in cell growth and proliferation. Often acting as aptamers, these molecules interact with critical proteins that regulate the induction of apoptosis or necrosis. As described herein, a specialized class of ODNs that contain a monomeric sequence of guanosine nucleotides (i.e., G-rich oligonucleotides or GROs) are capable of inducing apoptosis in malignant cells, for example, esophageal cancer cells.

Experimental results described herein suggest that the GROs of the invention work by inducing retardation in the progression of the cell cycle and then by creating a subG1 population of apoptotic cells. The reaction is dose dependent and appears to rely on the capacity of the GROs to adopt a G-quartet conformation. Importantly, nonmalignant esophageal cells or normal human lung fibroblasts are not impeded in their cell cycle progression when incubated with the GROs of the invention.

The cell cycle consists of four distinct phases: G₁ phase, S phase, G₂ phase (collectively known as interphase) and M phase. M phase is itself composed of two tightly coupled processes: mitosis, in which the cell's chromosomes are divided between the two daughter cells, and cytokinesis, in which the cell's cytoplasm divides forming distinct cells. Activation of each phase is dependent on the proper progression and completion of the previous one. Cells that have temporarily or reversibly stopped dividing are said to have entered a state of quiescence called G₀ phase.

The relatively brief M phase consists of nuclear division (mitosis) and cytoplasmic division (cytokinesis). After M phase, the daughter cells each begin interphase of a new cycle. Although the various stages of interphase are not usually morphologically distinguishable, each phase of the cell cycle has a distinct set of specialized biochemical processes that prepare the cell for initiation of cell division.

The first phase within interphase, from the end of the previous M phase till the beginning of DNA synthesis is called G₁ (G indicating gap or growth). During this phase the biosynthetic activities of the cell, which had been considerably slowed down during M phase, resume at a high rate. This phase is marked by synthesis of various enzymes that are required in S phase, mainly those needed for DNA replication. Duration of G₁ is highly variable, even among different cells of the same species.

The ensuing S phase starts when DNA synthesis commences; when it is complete, all of the chromosomes have been replicated, i.e., each chromosome has two (sister) chromatids. Thus, during this phase, the amount of DNA in the cell has effectively doubled, though the ploidy of the cell remains the same. Rates of RNA transcription and protein synthesis are very low during this phase. An exception to this is histone production, most of which occurs during the S phase. The duration of S phase is relatively constant among cells of the same species.

The cell then enters the G₂ phase, which lasts until the cell enters the next round of mitosis. Again, significant protein synthesis occurs during this phase, mainly involving the production of microtubules, which are required during the process of mitosis. Inhibition of protein synthesis during G₂ phase prevents the cell from undergoing mitosis. The term “post-mitotic” is sometimes used to refer to both quiescent and senescent cells. Nonproliferative cells in multicellular eukaryotes generally enter the quiescent G₀ state from G₁ and may remain quiescent for long periods of time, possibly indefinitely (as is often the case for neurons). This is very common for cells that are fully differentiated.

Cellular senescence is a state that typically occurs in response to DNA damage or degradation that would make a cell's progeny nonviable; it is often a biochemical alternative to the self-destruction of such a damaged cell by apoptosis. Some cell types in mature organisms, such as parenchymal cells of the liver and kidney, enter the G₀ phase semi-permanently and can only be induced to begin dividing again under very specific circumstances; other types, such as epithelial cells, continue to divide throughout an organism's life.

Checkpoints are used by the cell to monitor and regulate the progress of the cell cycle. If a cell fails to meet the requirements of a phase it will not be allowed to proceed to the next phase until the requirements have been met. Several checkpoints are designed to ensure that damaged or incomplete DNA is not passed on to daughter cells. Two main checkpoints exist: the G1/S checkpoint and the G2/M checkpoint. G1/S transition is a rate-limiting step in the cell cycle and is also known as restriction point. An alternative model of the cell cycle response to DNA damage has also been proposed, known as the “postreplication checkpoint.”

The postreplication checkpoint is a eukaryotic cellular response that is triggered by the replication of genomic DNA which is damaged by spontaneous processes, chemical mutagens, or sunlight exposure. This response prevents cell cycle progression until postreplication repair processes are completed, and may control the activity of these DNA repair pathways. The postreplication checkpoint makes time for repair by delaying the onset of mitosis.

The chkl gene is required to mediate the postreplication checkpoint and is conserved in yeast and humans. Fission yeast cells in which the chkl gene has been disrupted progress normally through the cell cycle after exposure to UV radiation until they have carried damaged DNA through S-phase and the subsequent mitosis, at which point cells begin to die and exhibit gross chromosomal damage. The BRCAl tumor suppressor plays a role in the activation of human chkl, therefore the postreplication checkpoint may prevent the genetic changes that lead to cancer.

As used herein, “Guanosine” is a nucleoside, C₁₀H₁₃N₅O₅ comprising guanine attached to a ribose (ribofuranose) ring via a β-N₉-glycosidic bond. Guanosine can be phosphorylated to become GMP (guanosine monophosphate), cGMP (cyclic guanosine monophosphate), GDP (guanosine diphosphate) and GTP (guanosine triphosphate). When guanine is attached to a deoxyribose ring, it is known as a deoxyguanosine.

Synthetic DNA molecules of a specific base sequence serve to block cancer cell division and promote cancer cell death. The synthetic DNA molecules described herein are G-rich oligos (GROs), which have a homogeneous sequence of guanosine bases. The invention includes a nucleotide molecule consisting of an oligonucleotide sequence of 19-21 guanosine residues, and further a nucleotide molecule having about 20 guanosine residues in length. The DNA molecule set forth in SEQ ID NO. 1 is a 20 residue sequence of guanosine residues that is an embodiment of the present invention. This molecule forms a G-quartet structure characteristic of G-rich oligonucleotides (GROs). (G-quartet structures form when four G-bases associate in a cyclic Hoogsteen H-bonding arrangement with each G-base making two H-bonds with a neighboring G-base).

The GROs of the invention are characterized by a G-quartet structure. The DNA molecules of the invention promote apoptosis in cells and inhibits cell growth. Preferably, the molecules promote apoptosis and inhibit cell growth in cancer cells. Cancer cells include esophageal, colorectal, breast, prostate, lung, brain, and other carcinoma cells. Acute effectiveness for promoting cell death is demonstrated by molecules of the invention in esophageal carcinoma cells. Not all molecules forming a G-quartet structure demonstrate the ability to promote apoptosis that the present invention demonstrates.

Another embodiment of the invention is directed to a pharmaceutical composition generally comprising an oligonucleotide of SEQ ID NOs: 1-3; in a preferred embodiment the oligonucleotide has a sequence of 19-21 guanosine residues in length, and more specifically, a sequence 20 guanosine residues in length, such as the sequence set forth in SEQ ID 1. The pharmaceutical composition further comprises an appropriate pharmaceutical adjuvant. For an injectable pharmaceutical composition, the DNA molecule may be dissolved in a PEG buffer solution, or other suitable known solutions. Such an injectable pharmaceutical may be appropriate for administration directly into a cancer tumor, a region approximate to a tumor, or alternatively into a patient's bloodstream. In other applications, the pharmaceutical composition comprises an appropriate pharmaceutical carrier to facilitate delivery of the oligonucleotides. The use and choice of carriers and adjuvants may be selected to optimize delivery of the oligonucleotide for efficacious treatment, and will be described in further detail.

Another embodiment of the invention includes a method of treating cancer comprising administering an effective amount of a pharmaceutical composition comprising an oligonucleotide sequence of SEQ ID NOs. 1-3; in a preferred embodiment the oligonucleotide has 19-21 guanosine residues in length to a subject in need thereof. As used herein, the term “subject” can mean a cell, population of cells, in vitro, in vivo, or ex vivo; and/or an individual or patient, for example, a mammal such as a human. In another preferred embodiment, the method comprising administering a pharmaceutical composition having an effective amount of an oligonucleotide having 20 guanosine residues in length, such as the sequence set forth in SEQ ID 1, to an individual in need thereof.

Another embodiment of the invention includes a cellular protein and DNA molecule complex. This complex of protein bound to or interacting with a DNA molecule, such as the guanosine sequence described herein, may comprise a cellular protein interacting with a DNA molecule comprising the sequence set forth in SEQ ID NO. 1. This protein DNA complex may form inside a cell, and formation of the complex induces cancer cell death. In certain embodiments, the GRO of the invention is labeled with a chemical compound, or peptide that allows for its detection.

Due to the discovery that the GROs of the invention inhibit the cell cycle progression and induce apoptosis selectively in cancer cells, the GRO-protein complex can be useful as a diagnostic for the existence of a disease condition, for example, cancer. As such, the present invention also relates to methods for diagnosing cancer comprising administering the GRO of the invention and detecting for the presence of the GRO-protein complex. A multitude of detection means and assays exist in the art and include, by way of example only, the use of one or more of an antibody or antibody fragment that binds an epitope specific for the GRO-protein complex, biotin labeled GRO, fluorescently tagged GRO, or the like. In an embodiment of the invention, the labeled GRO is administered to a cell or patient and a population of cells is isolated and lysed. The cellular lysate can be analyzed using a fractionation means, for example, a column comprising a receptor for the GRO or labeled GRO, followed by detection by Western blot of the column-bound fraction.

By way of example, after administering the GROs to a cell or patient, the GRO-protein complexes can be isolated using an ion exchange media, for example, anion exchange, such as DEAE; or streptavidin-bound beads can be used to selectively isolate biotin-labeled GROs. In either case, the GRO-protein complexes can be eluted and the bound protein, if any, can be identified using antibody reporter assays, for example, ELISA or Western blot.

Exemplary oligonucleotide sequences of the invention include:

HDG 20:

[SEQ ID NO: 1]

5′ GGGGGGGGGGGGGGGGGGGG 3′
NS 74nt/3S or 74nt/3S:

[SEQ IS NO: 2]

5′C*T*C*GTGCTTTCAGCTTCGATGTAGGAGGGCGTGGATACGTCCTGC
GGGTAAATAGCTGCGCCGATGGTTTC*T*A*C 3′
Active 26nt sequence (no modifications):

[SEQ ID NO: 3]

5′ GAATTCAGACAGTACCGGAATGCC 3′

In the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”). Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

As used herein, “nucleic acid molecule” is meant to refer generally to a series of linked nucleotide residues, and includes oligonucleotide and polynucleotide molecules. The nucleic acid can be single, double, or multiple stranded and can comprise modified or unmodified nucleotides or non-nucleotides or various mixtures and combinations thereof. A short oligonucleotide sequence may be based on, or designed from, a genomic, cDNA, or RNA sequence. Oligonucleotides of the invention comprise a nucleic acid sequence having about 10 nt, 50 nt, 100 nt or 200 nt in length, preferably about 19 nt to 200 nt in length. In certain embodiments of the invention, an oligonucleotide comprising a nucleic acid molecule less than 200 nt in length would further comprise the oligonucleotides of SEQ ID NOS: 1-3.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

By “nucleotide” is meant a heterocyclic nitrogenous base in N-glycosidic linkage with a phosphorylated sugar, and includes ribonucleotides as well as deoxyribonucleotides and analogs, mimetics, and derivatives thereof. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleobases known in the art as summarized by Limbach et al., 1994, Nucleic Acids Res. 22, 2183. Some of the non-limiting examples of chemically modified and other natural nucleic acid bases that can be introduced into nucleic acids include, for example, peptide nucleic acids, phosphorothioate, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “vectors” is meant any nucleic acid-based technique used to deliver a desired nucleic acid, for example, bacterial plasmid, viral nucleic acid, HAC, BAC, and the like.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, rats, mice, dogs, and cats. The cell can be prokaryotic (e.g., bacterial cell) or eukaryotic (e.g., mammalian or plant cell).

Oligonucleotides (eg; antisense, GeneBlocs) are synthesized using protocols known in the art as described in Caruthers et al., 1992, Methods in Enzymology 211, 319, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163). While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above.

The oligonucleotides can be introduced into cells or tissues by any technique known to one of skill in the art. Such techniques include, for example, electroporation, liposome transfer, naked nucleic acid insertion, particle bombardment and calcium phosphate precipitation.

In one embodiment the transfection is performed with a liposomal transfer compound, for example, DOTAP (N-1-(2,3-Dioleoyloxy)propyl-N,N,N-trimethyl-ammonium methylsulfate, Boehringer-Mannheim) or an equivalent, such as LIPOFECTIN®. Other liposomal transfer compounds include, for example, Lipofectamine® and Superfect®. In another embodiment, the transfection technique uses cationic lipids. Other methods include the use of macromolecular carriers, including an aqueous-cored lipid vesicle or liposome wherein the oligonucleotide is trapped in the aqueous core. Such vesicles are made by taking a solvent-free lipid film and adding an aqueous solution of the oligonucleotide, followed by vortexing, and extrusion or passage through a microfiltration membrane. In one embodiment the lipid constituents are a mixture of dioleoyl phosphatidylcholine/dioleoyl phosphatidylserine/galactocerebroside at a ratio of 1:1:0.16. Other carriers include polycations, such as polyethylenimine, having a molecular weight of between 500 daltons and 1.3 Md, with 25 kd being a suitable species and lipid nanospheres, wherein the oligonucleotide is provided in the form of a lipophilic salt.

The methods of the invention can be used with a wide range of concentration of oligonucleotides. For example, good results can be achieved with 10 nM/10⁵ cells. The transfected cells may be cultured in different media, including, for example, in serum-free media, media supplemented with human serum albumin or human serum.

“Aptameric” oligonucleotides are oligonucleotide molecules that bind a specific target molecule such as small molecules, proteins, nucleic acids, and even cells, tissues and organisms. Aptamers offer the utility for biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

Contemplated by the invention are also oligonucleotides containing one or more nucleotide derivatives or analogs. Nucleotide “derivatives” are modified nucleic acid sequences formed from the native compounds either directly, by modification, or by partial substitution. “Analogs” are nucleic acid sequences or amino acid sequences that have a structure similar to, but not identical to, the native compound, e.g. they differ from it in respect to certain components or side chains. Analogs may be synthetic or derived from a different evolutionary origin and may have a similar or opposite metabolic activity compared to wild type. Several exemplary types of nucleotide derivatives and analogs, which are contemplated as being useful in the present invention, are described in detail below.

Phosphodiester Moiety Analogs. Numerous analogs to the naturally occurring phosphodiester backbone have been used in oligonucleotide design. Phosphorothioate, phosphorodithioate, and methylphosphonate are readily synthesized using known chemical methods. Because novel nucleotide linkages can be synthesized manually to form a dimer and the dimer later introduced into the oligonucleotide via automated synthesis, the range of potential backbone modifications is as broad as the scope of synthetic chemistry. For example, the oligonucleotide may be substituted or modified in its internucleotide phosphate residue with a thioether, carbamate, carbonate, acetamidate or carboxymethyl ester.

Unlike the naturally occurring phosphodiester moieties, many phosphodiester analogs have chiral centers. For example, phosphorothioates, methylphosphonates, phosphoramidates, and alkyl phosphotriesters all have chiral centers. One skilled in the art would recognize numerous other phosphodiester analogs that possess chiral centers. Because of the importance of stereochemistry in hybridization, the stereochemistry of phosphodiester analogs can influence the affinity of the oligonucleotide for its target.

Most phosophodiester backbone analogs exhibit increased resistance to nuclease degradation. In an embodiment, phosphorothioates, methyl phosphonates, phosphorimidates, and/or phosphotriesters are used to achieve enhanced nuclease resistance. Increased resistance to degradation may also be achieved by capping the 5′ and/or 3′ end of the oligonucleotide. In an embodiment, the 5′ and/or 3′ end capping of the oligonucleotide is via a 5′--5′ and/or 3′--3′ terminal inverted linkage.

Phosphorothioate oligodeoxynucleotides are relatively nuclease resistant, water soluble analogs of phosphodiester oligodeoxynucleotides. These molecules are racemic, but still hybridize well to their RNA targets. Stein, C., et al. (1991) Pharmac. Ther. 52:365 384. Phosphorothioate oligonucleotides may be stereo regular, stereo non-regular or stereo random. A stereo regular phosphorothioate oligonucleotide is a phosphorothioate oligonucleotide in which all of the phosphodiester linkages or phosphorothiodiester linkages polarize light in the same direction. Each phosphorous in each linkage may be either an Sₚ or Rₚ diastereomer.

Sugar Moiety Analogs. Oligonucleotide analogs may be created by modifying and/or replacing a sugar moiety. The sugar moiety of the oligonucleotide may be modified by the addition of one or more substituents. For example, one or more of the sugar moieties may contain one or more of the following substituents: amino-alkylamino, aralkyl, heteroalkyl, heterocycloalkyl, aminoalkylamino, 0, H, an alkyl, polyalkylamino, substituted silyl, F, Cl, Br, CN, CF₃, OCF₃, OCN, O-alkyl, S-alkyl, SOMe, SO₂Me, ONO₂, NH-alkyl, OCH₂CH═CH₂, OCH₂CCH, OCCHO, allyl, O-allyl, NO₂, N₃, and NH₂.

Modification of the 2′ position of the ribose sugar has been shown in many instances to increase the oligonucleotide's resistance to degradation. For example, the 2′ position of the sugar may be modified to contain one of the following groups: H, OH, OCN, O-alkyl, F, CN, CF₃, allyl, O-allyl, OCF₃, S-alkyl, SOMe, SO₂Me, ONO₂, NO₂, N₃, NH₂, NH-alkyl, or OCH═CH₂, OCCH, wherein the alkyl may be straight, branched, saturated, or unsaturated.

In addition, the oligonucleotide may have one or more of its sugars modified and/or replaced so as to be a ribose or hexose (i.e. glucose, galactose). Further, the oligonucleotide may have one or more modified sugars. The sugar may be modified to contain one or more linkers for attachment to other chemicals such as fluorescent labels. In an embodiment, the sugar is linked to one or more aminoalkyloxy linkers. In another embodiment, the sugar contains one or more alkylamino linkers. Aminoalkyloxy and alkylamino linkers may be attached to biotin, cholic acid, fluorescein, or other chemical moieties through their amino group.

Base Moiety Analogs. In addition, the oligonucleotide may have one or more of its nucleotide bases substituted or modified. In addition to adenine, guanine, cytosine, thymine, and uracil, other bases such as inosine, deoxyinosine, hypoxanthine may be used. In addition, isoteric purine 2′deoxy-furanoside analogs, 2′-deoxynebularine or 2′deoxyxanthosine, or other purine or pyrimidine analogs may also be used. By carefully selecting the bases and base analogs, one may fine tune the binding properties of the oligonucleotide. For example, inosine may be used to reduce hybridization specificity, while diaminopurines may be used to increase hybridization specificity.

Adenine and guanine may be modified at positions N3, N7, N9, C2, C4, C5, C6, or C8 and still maintain their hydrogen bonding abilities. Cytosine, thymine and uracil may be modified at positions N1, C2, C4, C5, or C6 and still maintain their hydrogen bonding abilities.

Some base analogs have different hydrogen bonding attributes than the naturally occurring bases. For example, 2-amino-2′-dA forms three, instead of the usual two, hydrogen bonds to thymine (T). Examples of base analogs that have been shown to increase duplex stability include, but are not limited to, 5-fluoro-2′-dU, 5-bromo-2′-dU, 5-methyl-2′-dc, 5-propynyl-2′-dC, 5-propynyl-2′-dU, 2-amino-2′-dA, 7-deazaguanosine, 7-deazadenosine, and N2-Imidazoylpropyl-2′-dG.

Pendant Groups. A “pendant group” may be linked to the oligonucleotide. Pendant groups serve a variety of purposes which include, but are not limited to, increasing cellular uptake of the oligonucleotide, enhancing degradation of the target nucleic acid, and increasing hybridization affinity. Pendant groups can be linked to any portion of the oligonucleotide but are commonly linked to the end(s) of the oligonucleotide chain. Examples of pendant groups include, but are not limited to: acridine derivatives (i.e. 2-methoxy-6-chloro-9-aminoacridine); cross-linkers such as psoralen derivatives, azidophenacyl, proflavin, and azidoproflavin; artificial endonucleases; metal complexes such as EDTA-Fe(II), o-phenanthroline-Cu(I), and porphyrin-Fe(II); alkylating moieties; nucleases such as amino-1-hexanolstaphylococcal nuclease and alkaline phosphatase; terminal transferases; abzymes; cholesteryl moieties; lipophilic carriers; peptide conjugates; long chain alcohols; phosphate esters; amino; mercapto groups; phenolic groups, radioactive markers; nonradioactive markers such as dyes; and polylysine or other polyamines.

In any of the embodiments described herein, the aptameric GRO of the invention can contain one or more of the nucleotide modifications described above.

Cellular Uptake. To enhance cellular uptake, the oligonucleotide may be administered in combination with a carrier or lipid. For example, the oligonucleotide may be administered in combination with a cationic lipid. Examples of cationic lipids include, but are not limited to, lipofectin, dotma, dope, DMRIE and DPPES. The oligonucleotide may also be administered in combination with a cationic amine such as poly (L-lysine). Oligonucleotide uptake may also be increased by conjugating the oligonucleotide to chemical moieties such as transferrin and cholesteryls. In addition, oligonucleotides may be targeted to certain organelles by linking specific chemical groups to the oligonucleotide. For example, linking the oligonucleotide to a suitable array of mannose residues will target the oligonucleotide to the liver.

The cellular uptake and localization of oligonucleotides may be monitored by using labeled oligonucleotides. Methods of labeling include, but are not limited to, radioactive and fluorescent labeling. Fluorescently labeled oligonucleotides may be monitored using fluorescence microscopy and flow cytometry.

The efficient cellular uptake of oligonucleotides is well established. For example, when a 20 base sequence phosphorothioate (PS) oligonulceotide was Injected into the abdomens of mice, either intraperitoneally (IP) or intravenously (IV). The oligonucleotide accumulated in the kidney liver, and brain. Chain-extended oligonucleotides were also observed. Argrawal, S., et al. (1988) Proc. Natl. Acad. Sci. U.S.A. 85:7079 7083. When the PS 27-oligonucleotide was given by IV to rats, the initial T_{1/2} (transit out of the plasma) was 23 min, while the T_{1/2}beta of total body clearance was 33.9 hours. The long beta half-life of elimination demonstrates that dosing could be infrequent and still maintain effective, therapeutic tissue concentrations. Iverson, P. (1991) Anti-Cancer Drug Des. 6:531.

Another aspect of the invention pertains to vectors, containing a GRO of the invention, for example, a nucleic acid having SEQ ID NOs: 1-3 operably linked with one or more transcription regulatory elements such that the GRO can be expressed transiently, stably, or via an inducible promoter system, e.g., IPTG, tetracycline, tissue specific, or the like. Numerous types of vectors are known by those of skill in the art and are expressly contemplated by the present invention.

As used herein, the term “vector” refers to a nucleic acid molecule capable of transporting another nucleic acid to which it has been “operably linked.” Within a recombinant expression vector, “operably-linked” is intended to mean that the nucleotide sequence of interest is linked to the regulatory sequence(s) in a manner that allows for transcription and/or expression of the nucleotide sequence (e.g., in an in vitro transcription/translation system or in a host cell when the vector is introduced into the host cell). One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription of sequences to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), and artificial chromosomes, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be transcribed.

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of transcription, and/or expression of protein desired, etc. The expression vectors of the invention can be introduced into host cells to thereby produce proteins or peptides, including fusion proteins or peptides, encoded by nucleic acids as described herein. The recombinant expression vectors of the invention can be designed for transcription and/or expression in prokaryotic or eukaryotic cells. For example, transcription and/or expression in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and/or translated in vitro, for example using T7 promoter regulatory sequences and T7 polymerase.

In another embodiment, the recombinant vector is capable of directing transcription of the GRO preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In other aspects, the invention relates to a host cell comprising the isolated GRO of the invention. In certain embodiments, the host cell comprises a vector, plasmid or artificial chromosome nucleic acid containing one or more transcription regulatory nucleic acid sequences operably linked with the GRO sequence of the invention. The vector or plasmid nucleic acids can be, for example, suitable for eukaryotic or prokaryotic cloning, amplification, or transcription. In other embodiments, the invention comprises a plurality of GRO sequences linked contiguously as a single polynucleotide chain. In still other embodiments, the invention comprises a nucleic acid vector containing a plurality of GRO sequences linked contiguously and operably linked with the nucleic acid sequence of the vector.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. For example, bacteria cells may be used to carry or clone nucleic acid sequences or express polypeptides. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce the GRO of the invention. In a preferred embodiment the host cell is eukaryotic cell, for example a human cell. In still other preferred embodiments, the cell is a human cancer cell, for example, a human esophageal cancer cell.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. Another method is to use a eukaryotic viral vector, such as simian virus 40 (SV40) or bovine papilloma virus, to transiently infect or transform eukaryotic cells. (Eukaryotic Viral Vectors, Cold Spring Harbor Laboratory, Gluzman ed., 1982). Preferably, a eukaryotic host is utilized as the host cell as described herein. The eukaryotic cell may be a yeast cell (e.g., Saccharomyces cerevisiae) or may be a mammalian cell, including a human cell.

Mammalian cell systems that utilize recombinant viruses or viral elements to direct expression may be engineered. For example, when using adenovirus expression vectors, the nucleic acid sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptides in infected hosts (e.g., Logan & Shenk, Proc. Natl. Acad. Sci. U.S.A. 81:3655-3659, 1984).

For long-term, high-yield production of recombinant genes, stable expression is preferred. Rather than using expression vectors that contain viral origins of replication, host cells can be transformed with the cDNA encoding an GRO controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci, which in turn can be cloned and expanded into cell lines. For example, following the introduction of foreign DNA, engineered cells may be allowed to grow for 1 to 2 days in an enriched media, and then are switched to a selective media. A number of selection systems may be used, including but not limited to the herpes simplex virus thymidine kinase (Wigler et al., Cell 11: 233, 1977), hypoxanthine-guanine phosphoribosyltransferase (Szybalska & Szybalski, Proc. Natl. Sci. U.S.A. 48: 2026, 1962), and adenine phosphoribosyltransferase (Lowy et al., Cell 22: 817, 1980) genes can be employed.

In certain embodiments the therapeutic GRO of the invention is complexed, bound, or conjugated to one or more chemical moieties to improve and/or modify, for example, bioavailability, half-life, efficacy, and/or targeting. In certain aspects of this embodiment, the GRO may be complexed or bound, either covalently or non-covalently with, for example, cationic molecules, salts or ions, lipids, glycerides, carbohydrates, amino acids, peptides, proteins, other chemical compounds, for example, phenolic compounds, and combinations thereof. In certain aspects the invention relates to a GRO of the invention conjugated to a polypeptide, for example, an antibody. In certain embodiments the antibody is specific for the protein or protein aggregate of interest and therefore targets the GRO to the protein and/or protein aggregate.

Therapeutic uses and formulations. The nucleic acids of the invention are useful in potential prophylactic and therapeutic applications implicated in a variety of disorders including, but not limited to: metabolic disorders, diabetes, obesity, infectious disease, anorexia, cancer, neurodegenerative disorders, Huntington's Disease, Alzheimer's Disease, Parkinson's Disorder, prion diseases (e.g., BSE and CJD), spinocerebellar ataxia, immune disorders, hematopoictic disorders, and the various dyslipidemias, metabolic disturbances associated with obesity, the metabolic syndrome X and wasting disorders associated with chronic diseases and various cancers, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, lupus erythematosus, obesity, transplantation, adrenoleukodystrophy, congenital adrenal hyperplasia, prostate cancer, neoplasm; adenocarcinoma, lymphoma, uterus cancer, fertility, leukemia, hemophilia, hypercoagulation, idiopathic thrombocytopenic purpura, immunodeficiencies, graft versus host disease, AIDS, bronchial asthma, rheumatoid and osteoarthritis, Crohn's disease; multiple sclerosis, treatment of Albright Hereditary Ostoeodystrophy, esophageal cancer, and other diseases, disorders and conditions of the like.

Preparations for administration of the therapeutic complex of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

The nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

A therapeutically effective dose refers to that amount of the therapeutic complex sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, intravenous, intraperitoneal, parenteral or rectal administration.

Also disclosed according to the present invention is a kit or system utilizing any one of the methods, selection strategies, materials, or components described herein. Exemplary kits according to the present disclosure will optionally, additionally include instructions for performing methods or assays, packaging materials, one or more containers which contain an assay, a device or system components, or the like.

While preferred embodiments of the invention have been shown and described herein, it will be understood that such embodiments are provided by way of example only. Numerous variations, changes and substitutions will occur to those skilled in the art without departing from the spirit of the invention in view of the present description and examples. Accordingly, it is intended that the appended claims cover all such variations as fall within the spirit and scope of the invention.

### EXAMPLES

### Example 1

### Interpretation of Cell Cycle Profiles Generated by Flow Cytometry

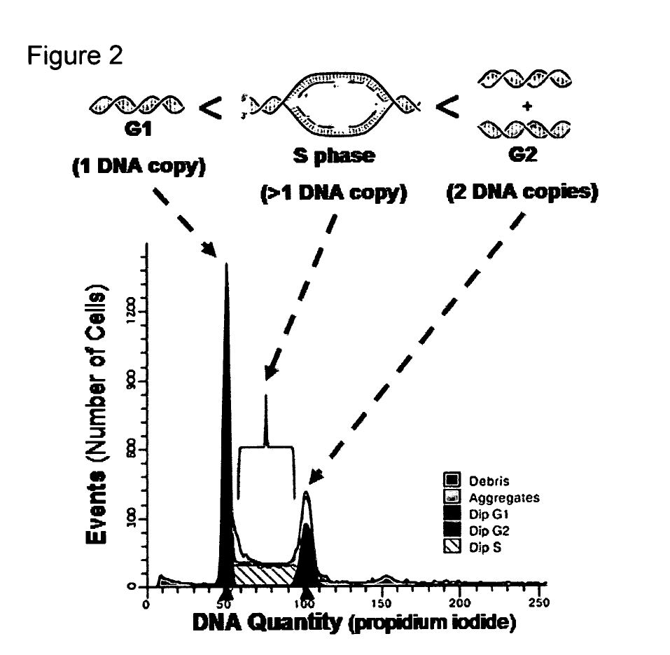

Cells are fixed at desired time-points post-transfection by cold ethanol incubation. Fixed cells are incubated for one hour with propidium iodide and RNase. The propidium iodide will bind to genomic DNA and is necessary for the subsequent quantification of total DNA in the cell; the RNase will eliminate any RNA from propidium iodide staining. Cells are subsequently analyzed by flow cytometry to visualize cell count versus propidium iodide content (as it directly relates to total DNA content). A typical cell cycle profile will generate two distinct peaks of cell populations. The first peak (G1 phase of the cell cycle) represents cells prior to any DNA replication, thus only having a singular content of diploid genomic DNA. A second distinct peak represents cells post-replication that contain two full contents of genomic DNA and are thus in cellular G2 phase. Cells which have DNA content greater than one diploid copy but less than a full two copies are in the process of DNA replication and are thus in cellular S phase (the region observed between the two distinct peaks). In the event of cellular apoptosis, genomic DNA will be degraded and the total DNA quantification will be less than one total DNA content and these cells will be observed prior to the G1 peak and interpreted as sub-G1/apoptotic cells. (See FIG. 2)

### Example 2

### OE19 Transfection with HDG20 Oligonucleotide

OE19 cells are grown in RPMI 1640+10% FBS to 50-70% confluency in 100 mm tissue culture dish. All growth media is then aspirated. Cells are carefully washed with 1 ml PBS so as not to detach cells, and then BPS is aspirated. 1 ml trypsin is added to dish and incubated at room temperature for 3-5 min. 1-2 ml of complete media is then added and pipetted up and down to release the cells. The cells are then transferred to a conical tube and quantified. Thereafter, the conical tube is spun at 1500 rpm for 5 min. All media is then aspirated from conical tube, leaving cell pellet. The pellet is resuspended in 100 μl RPMI (no serum) and transferred to an electroporation cuvette (4 mm gap). Experimental concentration of oligonucleotide (1-10 μM for HDG20) is then added, mixed well and electroporated (conditions: 250V, 2 pulse, 13 ms, Is interval). The contents of the cuvette are placed into 100 mm dish with 7 ml complete media and incubated for a desired time (96 hrs for HDG20 experimental conditions).

### Example 3

### Cell Cycle Analysis of Esophegeal Carcinoma Cells

Cells are trypsinized, spun and resuspended in 500 μl PBS. 5 ml 70% cold ethanol is added drop wise while vortexing gently. The mixture is then left on ice for 1 hour (and can be kept up to a few days at 4° C.). Cells are then spun for 5 min at 2000 rpm, washed one time with cold PBS and re-spun. The pellet is resuspended in FACS buffer (300 μl to 1 ml).

FACS buffer: 1 ml PBS; 20 μl RNase A 10 mg/ml (final concentration 50 μg/ml); 10 μl PI 1 mg/ml (final concentration 2.5 μg/ml); 10 μl FBS (1%).

Cells are then incubated at 37° C. for 1 hour while protected from light, and then incubated overnight at 4° C. before FACS analysis. In CellQuest->DNA QC->PBMC Experiment Document->Acquisition. 20,000 cells per sample are acquired; on FL2-A histogram, FL2 PMT Voltage is adjusted so the mean of the PBMC population is at channel 200 +/−5. Data is analyzed using ModFit LT software for FL-2 Area. Using auto-modeling, a histogram is created for each sample data and overlaid with graphical representations of the modeled G0/G1, S-phase, and G2/M. Histograms are fit to determine percentages of cells in each phase of the cell cycle.

### Example 4

Our experimental strategy is to examine the effect of oligonucleotides containing monomeric sequences of each of the four bases. Previous data implicate oligonucleotides containing guanosine residues, G-rich oligonucleotides (GROs), as potential inhibitors of cell growth (see above). Here, we electroporate OE19 cells, malignant esophageal cells and Het1A cells with GROs and ask if (1) progression through the cell cycle is altered, (2) if cell viability is affected and/or (3) if GROs exhibit a degree of specificity for either cell type. The oligonucleotides used in this study are 20-mers of monomeric sequence bearing no chemical modifications. Previous data indicate that GROs adopt a definable molecular structure known as a quadruplex or G-quartet. Such structures have a signatory pattern when examined by circular dichroism (CD); poly A, poly C and poly T molecules do not share such a distinctive footprint.

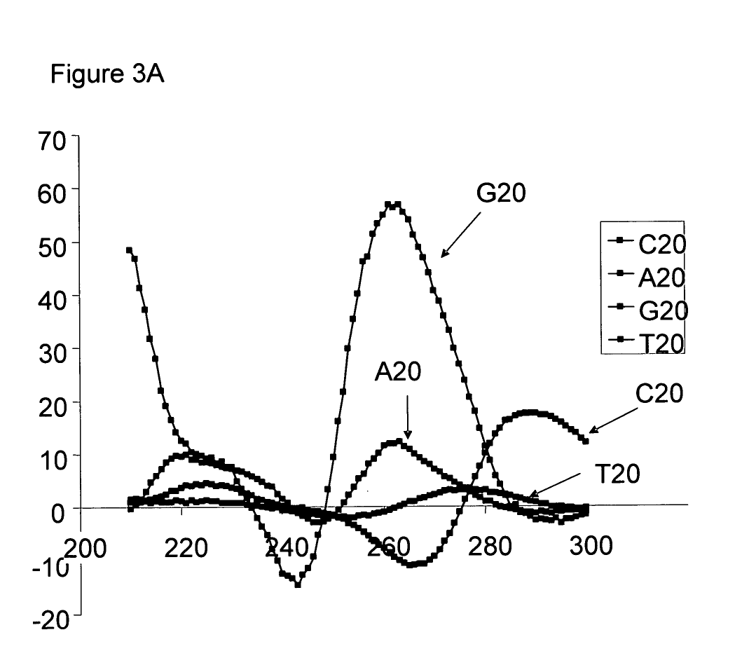

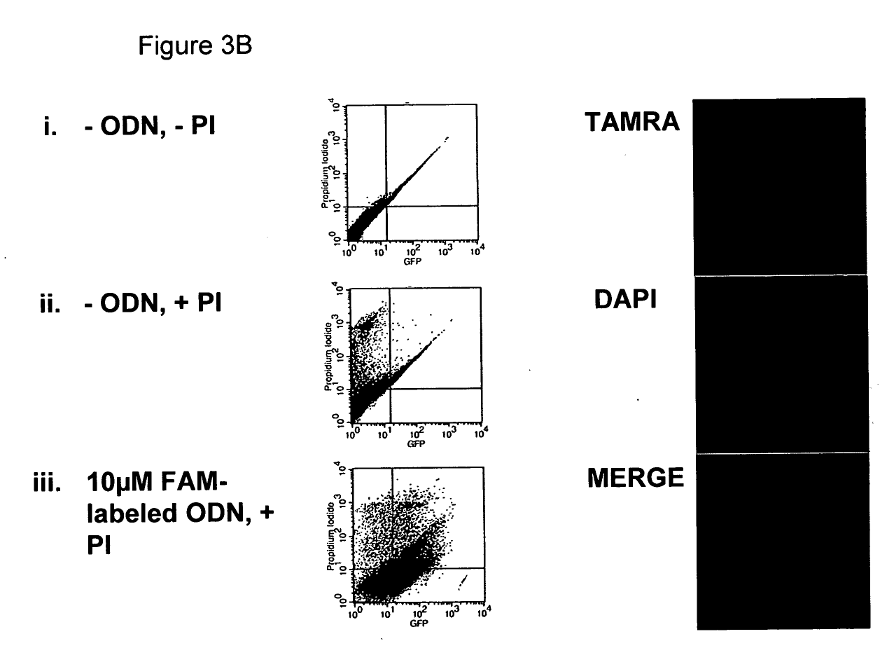

FIG. 3A presents the CD spectrum of each oligonucleotide used in this study and confirms that the monomeric GRO is in the quadruplex conformation. G-quartet forming oligos exhibit a maximal ellipticity 265 and a minimum ellipticity at 240. In comparison, none of the other oligonucleotides display evidence of a coordinated structure and under these reaction conditions are likely to be a heterogeneous population of random coils.

The significant degree of structural stability suggests that the GROs of the invention, for example, the monomeric guanosine oligonucleotide (SEQ ID NO. 1), have a stable G-quartet structure, a feature that has not previously been demonstrated for other “guanine-rich” oligonucleotides. As described herein, the structural stability appears to play an important role in mediating the biological activity of the GROs of the invention. While not being limited to any particular theory, the inventors believe that this structural stability mediates the aptameric function, i.e., the cellular protein or nucleic acid binding ability, and results in the functional efficacy, i.e., cancer cell cycle arrest and induction of apoptosis.

Therefore, the present invention also relates to the use of a GRO (for example, SEQ ID NO. 1 or 5) oligonucleotide as a “motif” or “module” within the context of a larger nucleic acid. For example, in certain embodiments the invention relates to nucleic acids comprising from 19-200 nucleotides, wherein the nucleic acid contains one or more GRO motifs, for example the G-rich motif of SEQ ID NO. 1 or 5. The incorporation of one or more GRO motifs can be useful for, for example, incorporation into an expression vector for transient or stable expression in a host cell. The cloning/subcloning of a short oligonucleotides, for example, monmeric G20 oligo (SEQ ID NO. 1) may be problematic, and therefore, use of a larger nucleic acid comprising the GRO motifs as modules can usurp the problems associated with subcloning of such a small homogenous oligo. As such, in a preferred embodiment the invention encompasses a nucleic acid molecule having from 19-200 nucleotides, wherein the nucleic acid comprises at least one G-rich motif, for example, the sequence of SEQ ID NO. 1.

Thus, based on the defined structure of the GRO 20-mer as a G-quartet, we decided to characterize the capacity of this molecule to affect cellular metabolism.

Oligonucleotide transfer into OE19 cells was examined using a TAMRA-labeled oligonucleotide. This fluorescent tag enables a tracing of the molecule as it enters the cell and provides an opportunity to quantitate uptake levels by FACS. We incubated 5 μM oligonucleotide with 10⁵ OE19 cells and examined uptake 24 hours later. As shown in FIG. 3C, electroporation of the FAM-labeled molecule produces transfer efficiencies in the range of 35-50% respectively. For these experiments, we combined fluorescence with viability by graphing fluorescence on the X-axis and exclusion of propidium iodide on the Y-axis. As such, cells remaining alive and containing FAM-labeled molecules appear in the lower right (LR) quadrant (FIG. 3C). Hence, 24 hours after electroporation, approximately 35-50% of OE19 cells contain TAMRA-labeled oligonucleotide. Previous data indicate that oligonucleotides (ODNs) uptake is not increased after 24 hours no matter what sequence is contained in the ODN.

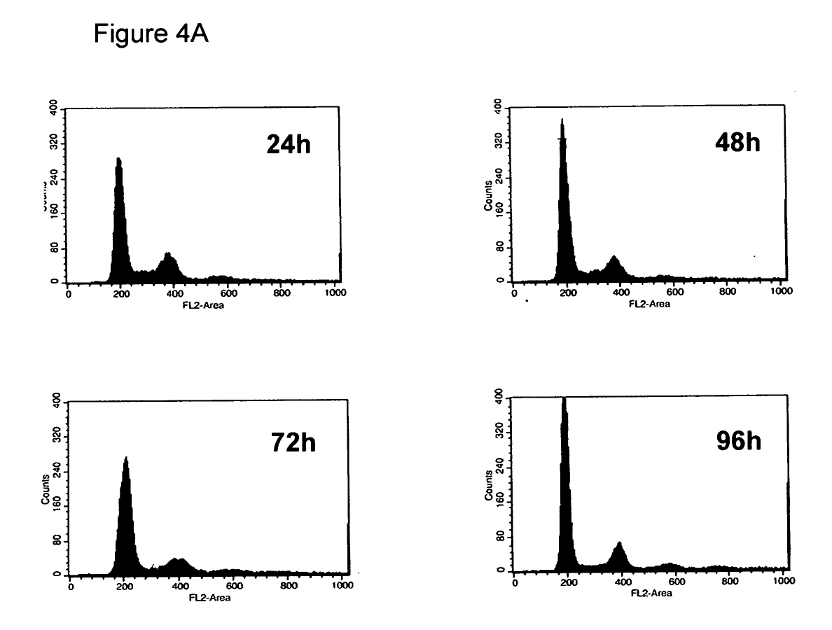

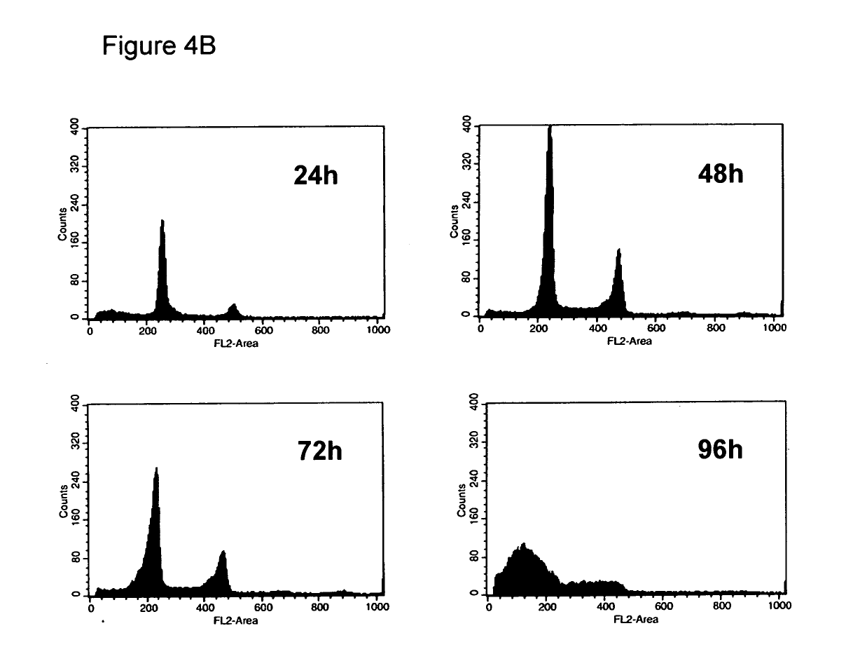

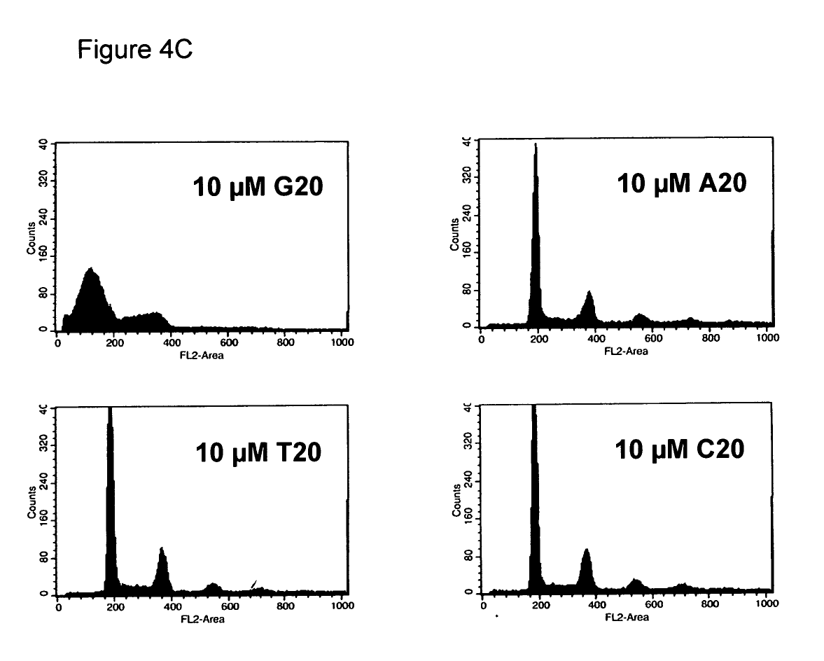

GROs have been reported previously to have antiproliferative effects on tumor cells presumably by influencing the population in such a way that more cells accumulate in S phase. To examine the influence of the monomeric 20-mer, GRO, we electroporated the molecule into OE19 cells and visualized the cell cycle profile at 24 hour intervals. FACS was also used to examine propidium-iodide stained nuclei of treated and mock-treated (lacking ODN) at each time point. As shown in FIGS. 4A and 4, the profiles of the treated and untreated populations are quite different. For the control samples, distinct G1, S and G2 phases are readily visible at each time point (FIG. 4A). In contrast, treated cells display a significant subG1 population as early as 48 hours post electroporation. The 24 hour point in both samples displays some subG1 debris due, in all likelihood, to cell death resulting from the electroporation process; this population disappears after 48 hours (see 48 hour point, FIG. 4B). A large subG1 population of cells appears at 72 hours in the treated sample and by 96 hours, the standard cell cycle profile is lost; the majority of cells are located at the far left of the spectra (sub G1 region). Cells with this type of profile usually have an apoptotic character. Since the 96 hour time point showed the most dramatic effect, we tested the other monomeric ODNs at this time point to address the question of specificity. OE19 cells were electroporated with 20-mers of G, T, A or C respectively at 1 μM and cell cycle profiles were taken once again at 96 hours. As shown in FIG. 4C, the population of cells treated with A20, T20 and C20 all exhibit a normal pattern while cells treated with G20 display a massive subG1 population. The percentage of cells in each phase from the A20, T20 and C20 treated samples is nearly identical (see data at right of each graph). These data suggest that the 20-mer monomeric GRO uniquely disrupts cell cycle progression resulting in the generation of a subG1, apoptotic population of OE19 cells after 96 hours of treatment.

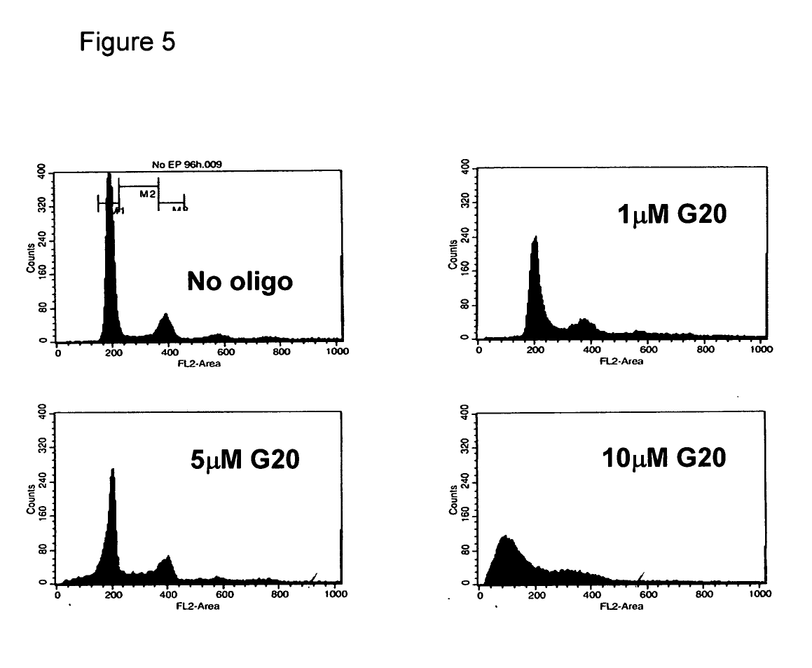

Finally, we wondered if the GRO effect could be seen in a dose dependent fashion. Hence, we electroporated OE19 cells with GRO at 1 μM, 5 μM, and 10 μM respectively and analyzed cell cycle profile 96 hour later. FIG. 5 shows that there is indeed a dose effect on the generation of the subG1 population. The influence of the GRO is apparent in cells treated at the 1 μM concentration. Here, we observe an accumulation of cells in each phase. Note that the G1 and G2 peaks are widened and there is an expansion of the zone representing the S phase population. This suggests that, although the cells are progressing through each phase of the cell cycle, they are spending a larger time in each region. At the 5 μM concentration, the phases again appear to be widened and the subG1 population in evident. At the 10 μM GRO level, the profile seen previously is evident with a large subG1 peak. Taken together, these data indicate that the G20 effect is dose-dependent and may be due to a lengthening of the time that cells spend in each phase of the cell cycle. Such retardation may activate apoptotic pathways that respond naturally to damage-induced cell stalling; the result of which is the evolution of a subG1 population.

### Example 5

### Induction of Apoptosis

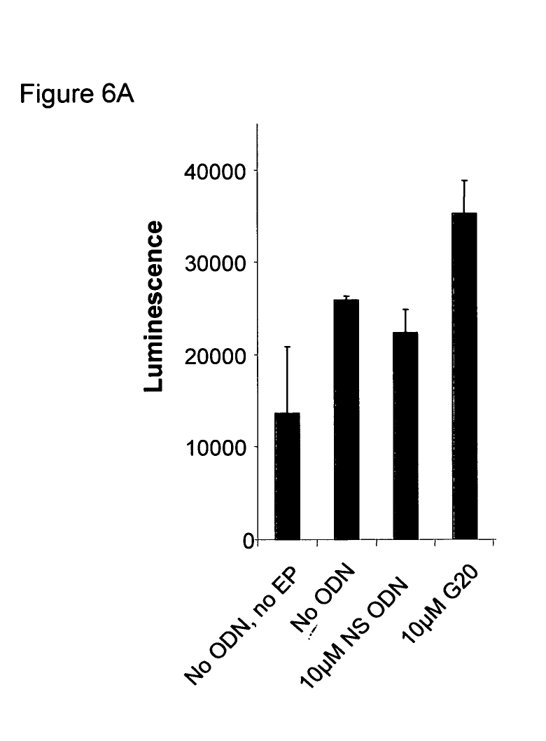

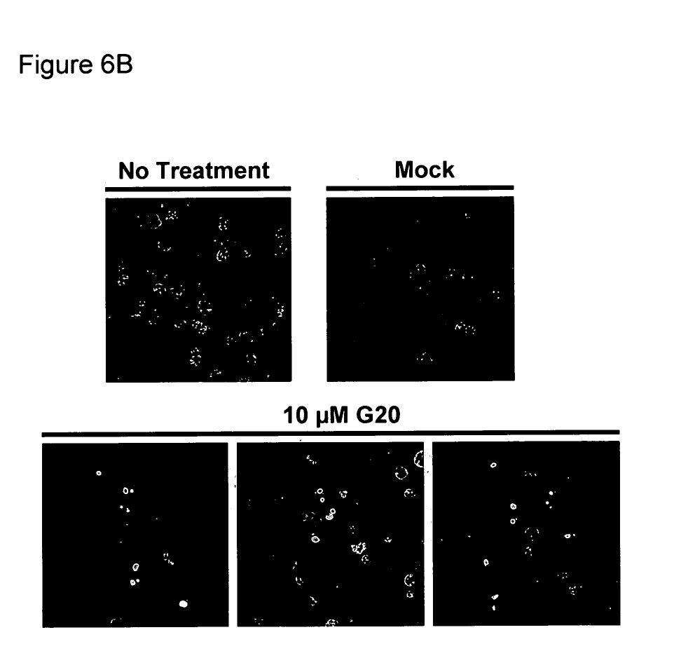

The appearance of the subG1 population suggested that an induction of apoptosis had taken place. To examine this question more directly, we carried out an assay that measures the activation of caspase 3 and caspase 7, both of which are early indicators of apoptotic character. In the assay used herein (see Methods and Materials), an increase in relative light units (luminescence) is indicative of enhanced caspase activity. OE19 cells (1×10⁶) were electroporated with two different ODNs; the first is a 25-mer bearing no G-rich regions (NS), the second is G20. Cells were incubated in the presence or absence of ODN for 96 hours, at which time the cells were processed for the caspase assay. As shown in FIG. 6A, G20 induces a statistically significant response in caspase 3 and 7 activity. The nonspecific 25-mer was used as a control to show that the assay does not generate positive results in the presence of any ODN. The no treatment control (No ODN, No EP) and the mock control (No ODN) serve to establish baseline and insure that electroporation itself cannot explain the level of activated caspase 3 and 7. We have also evaluated other monomeric sequences and consistent with the cell cycle results, no increase in apoptosis is observed (data not shown). Thus, we suggest that G20 induces some early stage apoptotic events when electroporated into OE19 cells. A late indicator of apoptosis and result of apoptotic signaling is the observation of chromatin condensation and fragmentation of cell nuclei. Cells were incubated in the presence or absence of ODN for 96 hours, stained with Hoechst 33342 nucleic acid dye, and imaged to assess for apoptotic nuclear morphology.

As shown in FIG. 6B, the no treatment control (No ODN, No EP) and the mock control (No ODN) show no discernable nuclear fragmentation. In contrast, clear chromatin condensation and nuclear fragmentation is observed in the G20-treated cells, correlating to the DNA fragmentation evidenced by flow cytometry at the same 96 hour time point. Thus, we further support that G20 induces apoptotic events when electroporated into OE19 cells.

### Example 6

### Specificity of G20 Activity

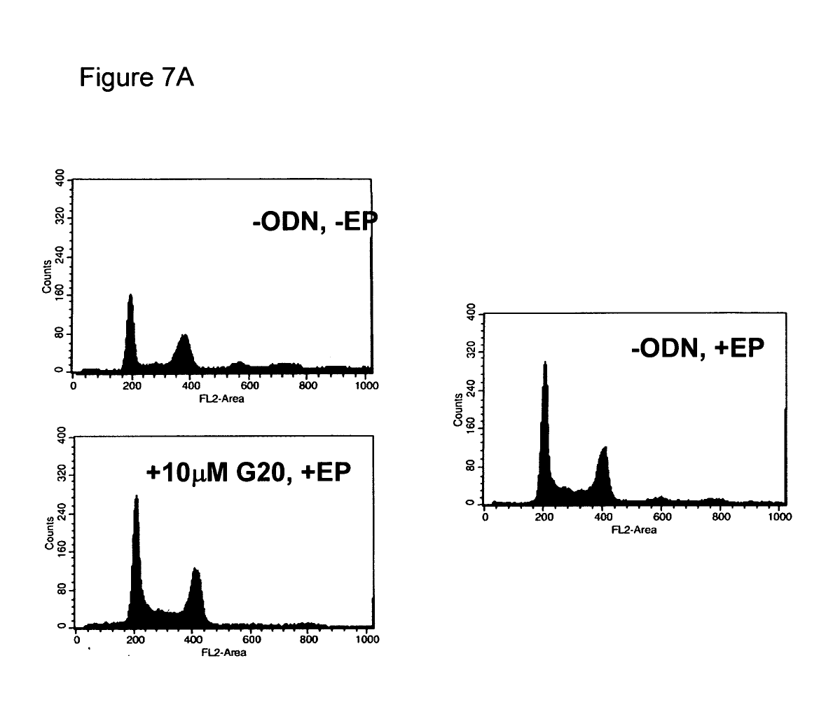

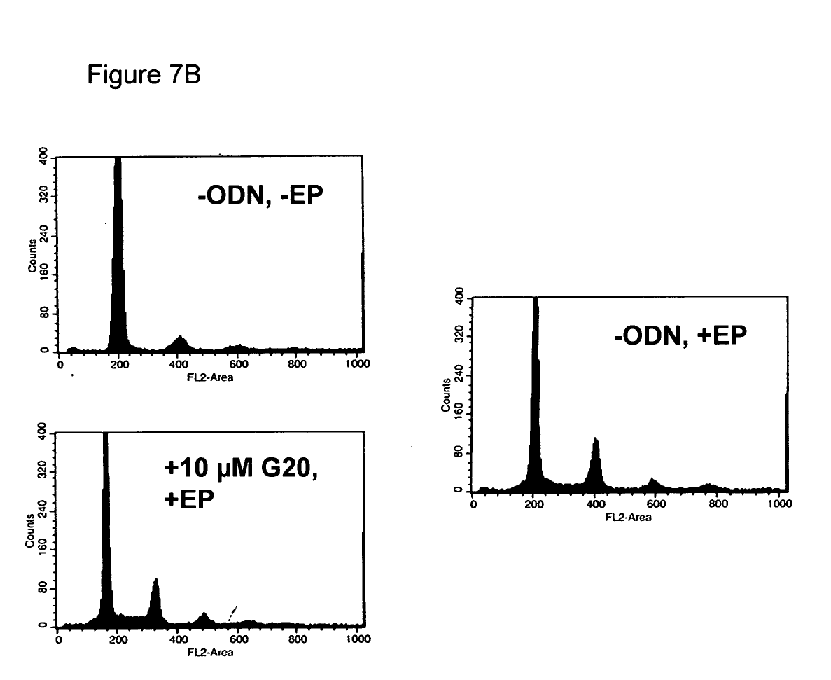

Our data suggest that addition of G20, a monomeric G-rich oligonucleotide, to a growing culture of OE19 cells induces the formation of subG1 population that is likely comprised of apoptotic cells. A question surrounding the importance of these observations centers on whether G20 has similar effects on a cell line that is not oncogenic. The Het-1A line arises from normal human esophageal mucosal cells and thus could serve as an important control. Thus, we asked if electroporation of 10 or 20 μM G20 resulted in an alteration of the normal cell cycle profile, specifically in the establishment of a subG1 population. Transfection efficiency of Het1A cell is approximately the same as the sister line OE19 maximizing at 35-40% (data not shown). After 96 hours of exposure to G20, the cell cycle profile was obtained and, as shown in FIG. 7A, exhibits no significant change. Most obvious is the lack of a subG1 population in the treated cells. The same is true for MRC5 cells, a nonmalignant human lung fibroblast cell line; in this case, 10 μM G20 or 20 μM G20 was electroporated into 2×10⁶ MRC5 cells and the cell cycle profile visualized 144 hours later (FIG. 7B). Again, no significant change is observed. Taken together, these data suggest that G20 appears to have some propensity for inducing the formation of a subG1 population (apoptotic) in OE19 cells with a measured level of specificity.

### Example 7

### Related G-Quartet ODNS and the Effect on OE19 Cell Proliferation

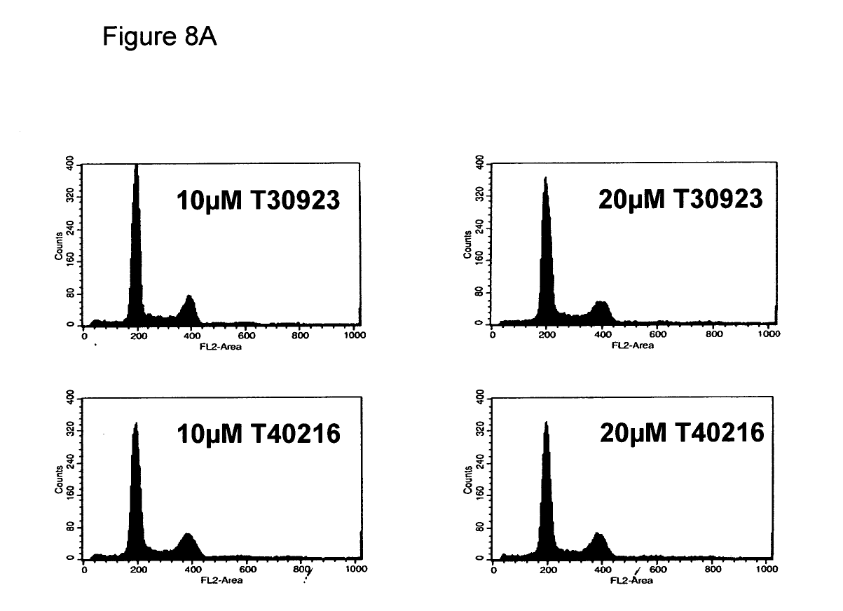

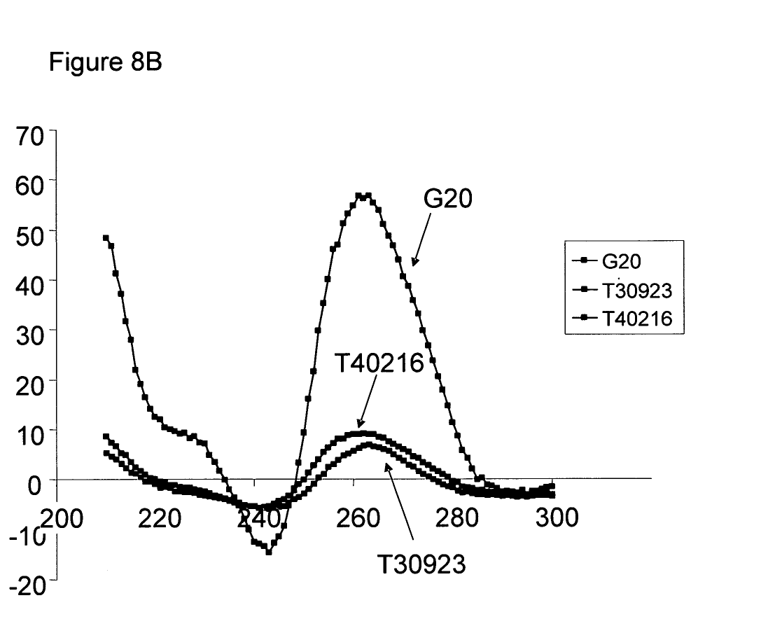

As shown in the results presented above, the monomeric GRO adopts a stable G-quartet structure (as judged by CD) and influences the proliferation of OE19 cells within the 96 hour time-frame. We questioned whether other ODNs that are known to adopt the G-quartet conformation in vitro exhibit the same activity. Thus, two other GROs were selected for addition to the OE19 cells under the same conditions and following the same protocol as with the G20 experiments. These two GROs are designated T30923 and T40216, T30923 is a 16-mer, bears the sequence (GGGT)₄ and contains two internal G-quartet structures as determined by NMR. Similarly, T40216 is also a 24-mer and contains the sequence (GGGGGT)₄; it also forms an intra-molecular G-quartet structure. Both of these GROs exhibit minimum ellipticity at 240 nm and maximum at 264 nm, characteristic of a G quartet. After introduction of either T30923 or T40216 into OE19 cells by electroporation, the cell cycle profiles were visualized at 96 hours. As shown in FIG. 8A, neither GRO induces the appearance of a significant subG1 population, certainly in sharp contrast to the impact of G20 (see FIG. 5).

To confirm that the T30923 and T40216 adopt the published structural conformation, we examined both of these ODNs by CD. As shown in FIG. 8B, both do, in fact, reflect the reported profile of G20 which exhibits a much higher value at 260 nm and sharper, more pronounced minima at 240 nm. This dramatic difference may indicate that the G20 molecule is in a more stable G-quartet conformation. As such, it may exhibit the specificity we observe in form of inhibition of cell proliferation and induction of apoptosis.

Single-stranded oligonucleotides (ODNs) that adopt a stable G quartet conformation can inhibit the proliferation of malignant esophageal OE19 cells in vitro. The mechanism by which OE19 cells are inhibited is apoptotic in nature and appears to be somewhat specific since neither non-malignant Het-1A esophageal cells nor breast MRC5 cells are affected in the same fashion. The antiproliferative effect can be readily seen by FACS analyses, displaying distinctive cell cycle profiles for each cell line. In a time dependent fashion, OE19 cells slow their growth and eventually amass into a subG1 population. This population contains elevated levels of caspase 3 and 7 and displays nuclear membrane breakdown; both of these cellular phenomenon are characteristic of cells undergoing apoptosis. The G rich ODNs (GROs) appear to be unique in this activity since the complementary monomeric A20, T20 and C20 do not promote apoptotic behavior. The G20 GRO is clearly the most stable molecule tested in this study as comparison GROs (20-4 and 20-6) bearing T residues within the 20-mer do not display the same activity. As shown in FIG. 8B, these two molecules, widely recognized as adopting a G quartet conformation appear less stable in the profiles generated by circular dichroism analyses.

GROs have been reported to play a role in multiple biological processes including the inhibition of human thrombin activity and integration of HIV. GROs have also been used in the design and development of telomerase inhibitors aimed at reducing oncogenic transformation. By and large, however, GROs have been investigated as non-antisense, antiproliferative agents that act to disrupt cell cycle progression. Their effect appears to be transduced through a cascade of reactions that likely include interactions with specific nuclear proteins. One of the major candidates is nucleolin, a protein with an extraordinary array of functions in cell growth and proliferation. Nucleolin can act as a structural component of the cell matrix while participating in DNA replication, cytokinesis and nuclear division. Bates and colleagues have demonstrated that certain types of GROs inhibit DNA helicase activity in vitro suggesting that these molecules arrest replication fork movement. Nucleolin has also been shown to localize in the plasma membrane perhaps functioning as a cell surface receptor.

Inhibition of cell proliferation through a blockage in DNA replication has been observed when a 29-mer bearing T residues placed after every 2 G residues (except for one spot where T residues are adjacent) is added to the cell culture. These studies were carried out in carcinoma cell lines. In OE19 cells, a similar molecule with the alternating GGT sequence displayed little inhibition of cell proliferation whereas the monomeric GRO (G20) induces a severe restriction on cell growth (see FIG. 8A). Thus, while both types of GROs are known to be in the G quartet structural conformation, it appears that the sensitivity of malignant cell lines to GROs in general can exhibit some variance. Our data suggest that the efficiency of inhibition may correlate with the degree of GRO stability as judged by circular dichroism.

It is also clear from previous work that antiproliferative effect is not primarily at the level protein synthesis, but more likely at the level of DNA replication. RNA synthesis takes place at the same level under conditions where DNA synthesis is aborted. Thus, it is possible that G20 exhibits its growth effect by interacting with nucleolin or another protein involved in DNA replication, e.g. RPA, etc. Because delivery of a GRO into the nucleus is problematic, the block most likely takes placed in the cytoplasm or as indicated above even at the plasma membrane. It is important to note that nucleolin protein functions in ribosome biogenesis and metabolic structuring, events that occur in cytoplasm. G20 may exert its antiproliferative effect by binding directly to nucleolin protein or an accessory protein that modulates the level of nucleolin available for nuclear activities involved in DNA replication.

### Example 8

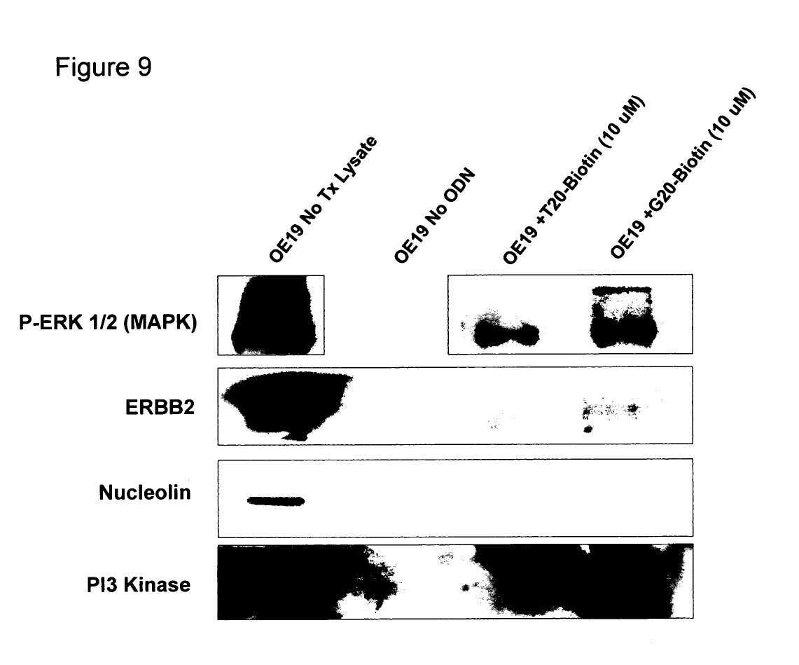

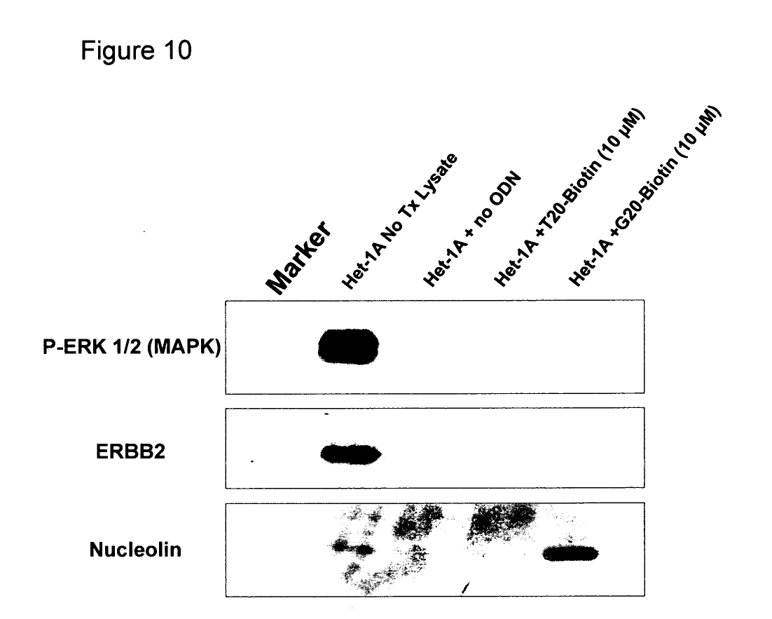

The GRO of the invention does not likely mediate its activity through nucleolin in cancer cells. In FIGS. 9 and 10, total protein from each cell line is isolated and incubated overnight with G20 single-strand deoxyoligonucleotide that contains a 5′ biotin conjugate. Thenstreptavidin beads were used to isolate proteins that bind to the biotin-labeled G20. The bound proteins are then identified using Western blot. It does not appear that G20 (SEQ ID NO. 1) binds to nucleolin in the malignant OE19 cell line, but that G20 does bind to nucleolin in the non-malignant Het-1A cell line. Therefore we do not believe that nucleolin is the cytotoxic/apoptotic factor in the malignant cell line, and we are currently trying to identify additional proteins that my be involved in the selective cell killing.

### Example 9

### OE19 Xenograft Study

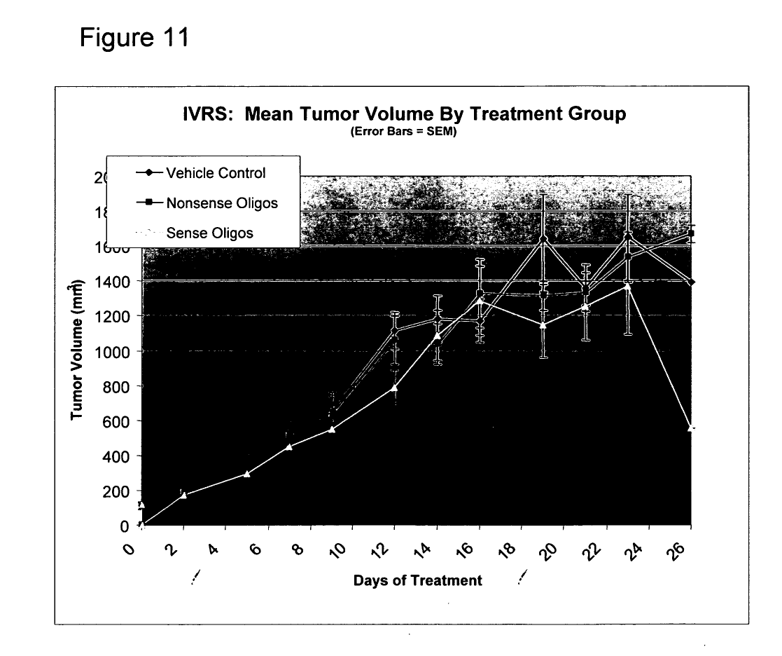

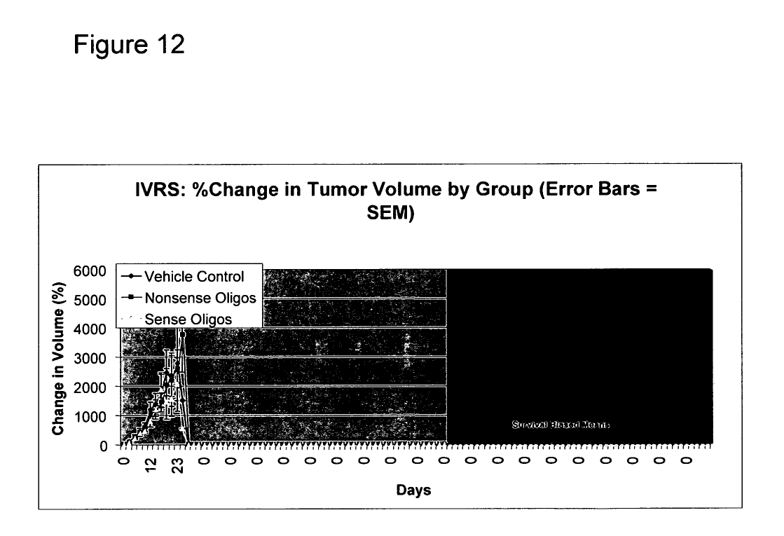

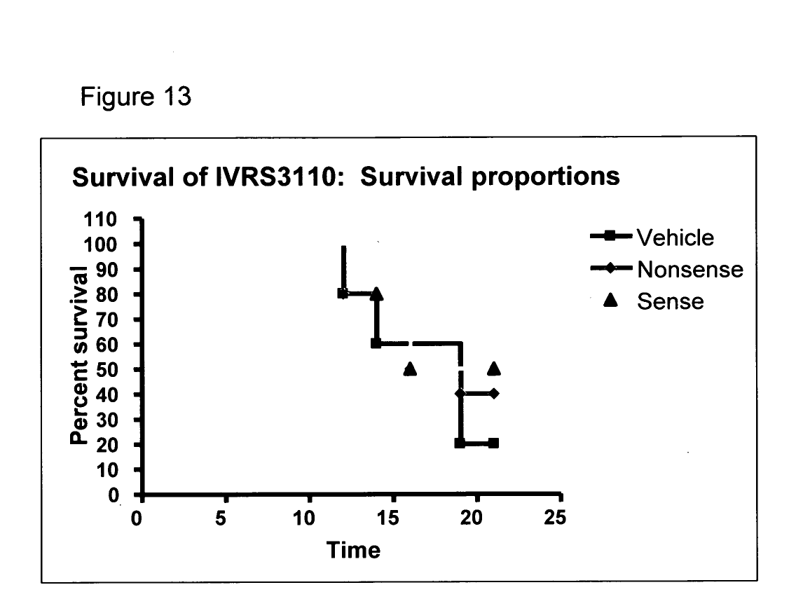

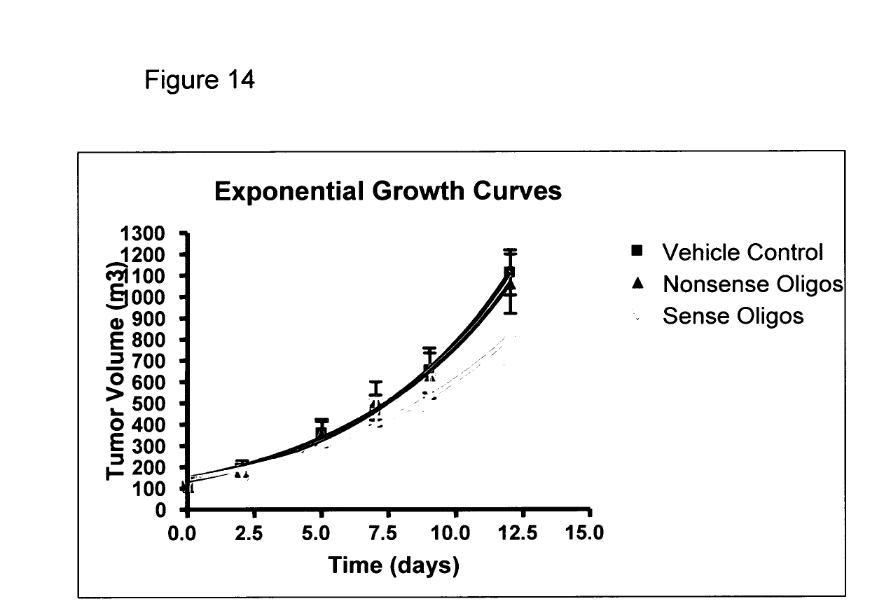

Mice were dosed with the oligonucleotide of SEQ ID NO. 1 and control oligonucleotides as follows. (See FIGS. 11-14). Following a 1 week acclimation 1×10⁶ OE19 cells grown in RPMI+L-glutamine+10% fetal bovine serum, were injected SC into the dorsal trunk. The tumor location was palpated up to daily to identify those animals with viable tumors. Once identified, the positive xenograft animals were divided in to vehicle, nonsense oligonucleotide, and sense oligonucleotide treatment cohorts.

The mice were treated twice during week 1 at day 1 and 3 (two treatments in total for each subject) by IV delivery of vehicle, sense oligonucleotide (10 mg/kg), or nonsense (10 mg/kg) oligonucleotide. Body weight was measured three times per week; Tumors were measured 3 times per week; and clinical observations were conducted three times per week.

The mice were sacrificed when tumor volume in 10% of the body weight or 1.5 cm in diameter and the tumors harvested, fixed, and analyzed. FIGS. 11-14 demonstrate that the in vivo administration of the G20 oligonucleotide (i.e., SEQ ID NO. 1) resulted in a significant decrease in tumor volume and an increase in animal viability in a dose dependent fashion. These results confirm the therapeutic utility of the present invention and also affirm the tumor selectivity of the GROs of the invention.

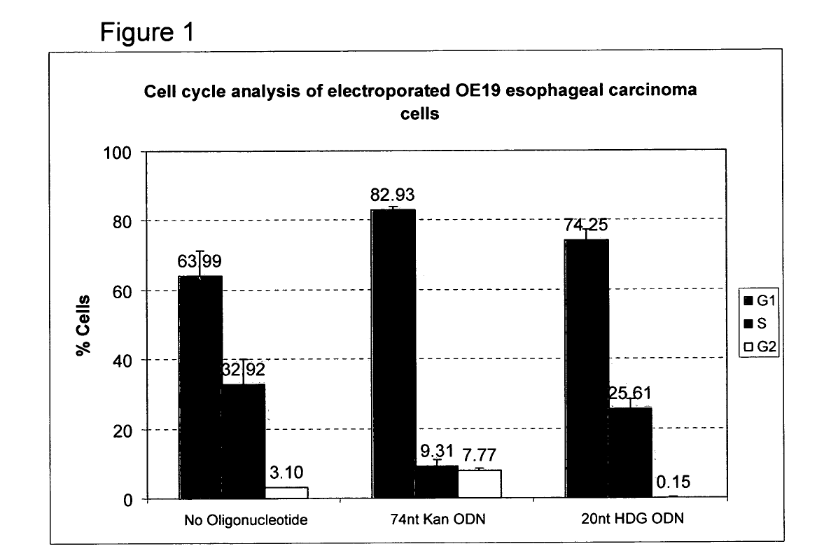

In an elegant series of studies, Tweardy and colleagues established STAT3 as a target for certain GROs. STAT3 upregulation takes placed during oncogenesis and may function as a mediator of oncogenic signaling. These workers demonstrate that GROs containing G sequences interrupted by T or C residues at regular intervals can bind to STAT3 and block its function as a transcription factor. In this case, it appears that their GROs are delivered directly into the nucleus by transfection using PEI. Again, in our hands and for OE19 cells, these GROs are much less effective in blocking cell proliferation. We did not, however, employ PEI as a transfection agent since it is toxic to most of our cell lines (data not shown). Thus, under our reaction conditions, and as shown in FIG. 1B, the majority of the G20 molecules locate in the cytoplasm proper. Hence, while we cannot rule out a direct block of transcription factor activity (and should not), we find that cytoplasmic targets are the more probable sites of antiproliferative activity in OE19 cells.

What is perhaps most intriguing about the inhibition of cell growth exhibited by G20 is its selectivity which is revealed at several levels; the first being the monomeric G sequence as opposed to strings of T, C or A respectively. A wealth of previous data certainly support the uniqueness of G20 activity found in our system; their antiproliferative effects are well documented. We know from structural studies with CD analyses that monomeric (all G) GROs are more stable that those that contain T or C residues interspersed throughout. In fact, an 11-mer, G₁₁T, was found to have a high degree of stability and has the capability of forming multimer structure known as a G-wire. Although we hypothesize that G20 is likely to assemble into this four-stranded, cage-like structure, we obviously do not know if a monomer or multimer GRO is the “active agent” in promoting apoptosis in OE19 cells. The mechanism of how a G-wire, a frayed wire or G-lego structure would preferentially exhibit anti-proliferative activity remains under investigation. The second level of selectivity is apparent in the drastic effect of G20 on the malignant OE19 esophageal line as compared to the nonmalignant Het1A esophageal cell line.

While significant differences in the expression of many genes is likely, one clear candidate emerges. The ERBB2 gene shares homology with the epidermal growth factor and exhibits tyrosine kinase activity. The ERBB2 protein participates in several signaling pathways which regulate cell growth and proliferation. It is also active in igniting apoptotic pathways which lead eventually to cell death. OE19 cells have been shown to have a 100-fold amplification of ERBB2 both at the genomic and mRNA level. This genetic change is not unique to OE19 cells as the amplification of ERBB2 is the most frequent genetic change in esophageal adenocarcinoma. As such, G20 may interact directly with ERBB2 and induce its apoptotic activity perhaps by binding to it at membrane or cytoplasmic sites. This suggestion is consistent with the data presented in FIG. 3B where the ODN is observed to localize predominantly in the cytoplasm; it may also be membrane bound. G20 may exert its influence in the same fashion as trastuzuarab (Herceptin) which acts as an antagonist to ERBB2. This drug reduced proliferation by blocking MAPK or PI3K pathways that are keys in regulating apoptosis.

Materials and Methods

Cell Line and Culture Conditions

The OE19 cell line was acquired from Peter Dahlberg (University of Minnesota). OE19 cells are grown in RPMI 1640 medium (Sigma-Aldrich, St. Louis, Mo.) with L-glutamine and 10% fetal bovine serum. The Het-1A cell line was acquired from ATCC (American Type Cell Culture, Manassas, Va.) and are grown in MEM Eagle medium with Earles salts, L-glutamine NaHCO₃, and 10% fetal bovine serum. The MRC5 cell line was acquired from ATCC (American Type Cell Culture, Manassas, Va.) and are grown in MEM Eagle medium with Earles salts, L-glutamine NaHCO₃, and 10% fetal bovine serum.

Oligonucleotides (ODNs) and Cell Electroporation Conditions

Oligonucleotides are synthesized by Sigma-Aldrich (St. Louis, Mo.). Cells grown in complete medium supplemented with 10% FBS and, where necessary, trypsinized and harvested by centrifugation. For electroporation, 2×10⁶ cells were resuspended in 100 μl serum-free medium and transferred to a 4 mm gap cuvette (Fisher Scientific, Pittsburgh, Pa.). The oligonucleotide was added at the desired final concentration and the cells were electroporated (250V, 13 ms, 2 pulses, 1s interval) using a BTX Electro Square Porator™ ECM 830 (BTX Instrument Division, Holliston, Mass.). The electroporated cells were then transferred to a 100 mm dish, recovered in complete medium supplemented with 10% FBS, and incubated at 37° C. for prior to experimental analysis.

Flow Cytometry Analysis

To analyze cell cycle profiles, transfected cells were seeded at a density of 1.5×10⁶ in a 100 mm dish in complete medium supplemented with 10% FBS. At the respective timepoints cells were trypsinized, resuspended in 300 μl cold PBS and fixed by addition of 700 μl cold 95% ethanol. Cells were incubated at 4° C. for 16 hours, subsequently washed and resuspended in 500 μl of PBS containing 50 μg/ml RNase A, 2.5 μg/ml propidium iodide, and 1% FBS and analyzed by a Becton Dickinson FACScalibur flow cytometer (Becton Dickinson, Franklin Lakes, N.J.) for DNA content. In addition, a TAMRA-labeled G20 oligonucleotide was used to measure transfection efficiency; fluorescence was quantitated by FACS analysis 24 hrs after electroporation in respective cell lines. Compartmentalization of the fluorescent oligonucleotide was imaged by confocal microscopy; nuclei are stained with DAPI.

Circular Dichroism Spectroscopy

Circular dichroism spectra of 15 μM oligonucleotide samples in 10 mM KCl were recorded on an AVIV model 202 spectrometer. Measurements were performed at 24° C. using a 0.1 cm path-length quartz cuvette (Hellma). The CD spectra were obtained by taking the average of two scans made at 1 nm intervals from 200 to 310 nm and subtracting the baseline value corresponding to that of buffer alone. Spectral data are expressed in units of millidegree.

Circular dichroism (CD) spectroscopy measures differences in the absorption of left-handed polarized light versus right-handed polarized light which arise due to structural asymmetry. The absence of regular structure results in zero CD intensity, while an ordered structure results in a spectrum which can contain both positive and negative signals. Secondary structure can be determined by CD spectroscopy in the “far-uv” spectral region (190-250 nm). At these wavelengths the signal arises when it is located in a regular, folded environment. The CD spectrum of in the “near-uv” spectral region (250-350 nm) can be sensitive to certain aspects of tertiary structure.

Caspase Assay

At respective timepoints treated cells were harvested and 5×10⁴ cells in 100 μL total volume was mixed with 100 μL of equilibrated Caspase-Glo 3/7 reagents (Promega, Madison, Wis.). After incubating at room temperature for 1 hour, samples were transferred to 96-well plate and luminescence was determined using a Wallac 1420 Victor³V micro-plate reader (PerkinElmer, Shelton, Conn.). Each data point represents three (+S.D.) independent experimental points.

Hoechst Staining

Chromatin condensation and nuclear fragmentation was visualized by Hoechst staining in OE19 cells at 96 hrs post electroporation. Cells treated with either non-specific or specific ODN (10 μM) or no ODN were grown in 8-chamber slides, and fixed with 2% paraformaldehyde, permeabilized with cold methanol, stained with Hoechst 33342 (Molecular Probes, (1:5000). The fields of cells were imaged at 40× magnification via confocal microscopy using a Ti:sapphire laser to detect Hoechst fluorescence.

Mice Studies

Thirty-six 5 to 7 week old male NODscid mice were ear notched for identification and housed 4 per cage. 1×10E60E19 cells were injected subcutaneously in to each mouse. The injection location was palpated to allow selection of those mice with identifiable tumor growth and these mice were divided in to vehicle, nonsense oligonucleotide, and sense oligonucleotide treatment groups. The mice were treated twice during week 1 at day 1 and 3 (two treatments in total for each subject) by IV delivery of vehicle (PBS), sense oligonucleotide (T20; 10 mg/kg), or nonsense (G20; 10 mg/kg) oligonucleotide. Body weights were taken three (3) times per week along with clinical observations and tumor measurement by caliper. Tumors were harvested once they reached 10% volume to body weight or 1.5 cm. Vehicle Control: Phosphate-Buffered Saline (PBS); Nonsense deoxyoligonucleotide: T20; Sense deoxyoligonucleotide: G20.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An oligonucleotide molecule having from 19 to 200 nucleotides comprising at least one G-quartet forming motif comprising 19-24 nucleotide residues wherein at least 83% of the residues are guanosine residues, and wherein the molecule inhibits cancer cell growth or proliferation and induces apoptosis in a cancer cell.

2. The oligonucleotide molecule of claim 1, wherein the oligonucleotide demonstrates a circular dichroism signal minimum at about 240 nm and a signal maximum at about 265 nm when measured at 24° C., in about 10 mM KCl at an oligonucleotide concentration of 15 μM.

3. The oligonucleotide molecule of claim 1, wherein the cancer cell is at least one of the types selected from the group consisting of esophageal, colorectal, breast, and prostate.

4. The oligonucleotide molecule of claim 1, wherein the molecule comprises the sequence set forth in SEQ ID NOs. 1 or 5 or combinations thereof.

5. The oligonucleotide molecule of claim 1, wherein the molecule consists of an oligonucleotide sequence of 19-21 guanosine residues.

6. The oligonucleotide molecule of claim 1, wherein at least one of the guanine residues is chemically modified.

7. The oligonucleotide molecule of claim 6, wherein the chemical modification comprises a biotin label.

8. The oligonucleotide molecule of claim 4, wherein the oligonucleotide is combined with at least one of a pharmaceutically acceptable excipient, carrier, adjuvant or combination thereof.

9. A method of preventing proliferation of a cancer cell comprising administering to a subject a composition comprising an effective amount of an oligonucleotide molecule having from 19 to 200 nucleotides comprising at least one G-quartet forming motif comprising 19-24 nucleotide residues wherein at least 83% of the residues are guanosine residues, and wherein the molecule inhibits cancer cell growth or proliferation and induces apoptosis in a cancer cell; and at least one of a pharmaceutically acceptable carrier, excipient, adjuvant or combination thereof.

10. The method of claim 9, wherein the molecule induces apoptosis in a cancer cell.

11. The method of claim 10, wherein the cancer cell is at least one of the types selected from the group consisting of esophageal, colorectal, breast, and prostate.

12. The method of claim 9, wherein the molecule comprises the sequence set forth in SEQ ID NOs. 1 or 5 or combinations thereof.

13. The method of claim 9, wherein the molecule consists of an oligonucleotide sequence of 19-21 guanosine residues.

14. The method of claim 9, wherein the molecule is administered in a pharmaceutically acceptable route selected from the group consisting of orally, parenterally, and combinations thereof.

15. A method of detecting/screening for cancer comprising administering a polynucleotide of SEQ ID NO. 1 to a subject; isolating and identifying any polynucleotide-protein complexes comprising a cellular protein and the polynucleotide of SEQ ID NO. 1.

16. The method of claim 15, wherein the cancer cell is at least one of the types selected from the group consisting of esophageal, colorectal, breast, and prostate.

17. The method of claim 15, wherein the polynucleotide-protein complex is identified using an antibody or antibody fragment that is capable of binding specifically to the polynucleotide-protein complex.

18. The method of claim 15, wherein the polynucleotide comprises a chemical modification.

19. The method of claim 18, wherein the chemical modification comprises a biotin label.

20. The method of claim 19, wherein the polynucleotide-protein complex is isolated using a streptavidin-conjugated matrix.


SEQUENCE LISTING (8 sequences)

SEQ ID NO: 1 - DNA, 20 bp, Artificial
  Synthetic oligonucleotide
  misc_feature (1)..(20): Synthetic Oligonucleotide
gggggggggggggggggggg

SEQ ID NO: 2 - DNA, 74 bp, Artificial
  Synthetic Oligonucleotide
  misc_feature (1)..(74): Synthetic Oligonucleotide
ctcgtgctttcagcttcgatgtaggagggcgtggatacgtcctgcgggtaaatagctgcgccgatggtttctac

SEQ ID NO: 3 - DNA, 24 bp, Artificial
  Synthetic oligonucleotide
  misc_feature (1)..(24): Synthetic oligonucleotide
gaattcagacagtaccggaatgcc

SEQ ID NO: 4 - DNA, 16 bp, Artificial
  Synthetic oligonucleotide
  misc_feature (1)..(16): Synthetic oligonucleotide
gggtgggtgggtgggt

SEQ ID NO: 5 - DNA, 24 bp, Artificial
  Synthetic Oligonucleotide
  misc_feature (1)..(24): Synthetic Oligonucleotide
gggggtgggggtgggggtgggggt

SEQ ID NO: 6 - DNA, 20 bp, Artificial
  Synthetic Oligonucleotide
  misc_feature (1)..(20): Synthetic Oligonucleotide
cccccccccccccccccccc

SEQ ID NO: 7 - DNA, 20 bp, Artificial
  Synthetic Oligonucleotide
  misc_feature (1)..(20): Synthetic Oligonucleotide
aaaaaaaaaaaaaaaaaaaa

SEQ ID NO: 8 - DNA, 20 bp, Artificial
  Synthetic Oligonucleotide
  misc_feature (1)..(20): Synthetic Oligonucleotide
tttttttttttttttttttt